(19) United States
(12) Patent Application Publication
(10) Pub. No.: US 2010/0273650 A1
(43) Pub. Date: Oct. 28, 2010

(54) Fungicidal Mixtures Comprising a Substituted 3-Hydroxymethylpyridine and a Further Fungicidal Compound

(75) Inventors: Jens Renner, Dirmstein (DE); Sarah Ulmschneider, Dirmstein (DE); Jochen Dietz, Karlsruhe (DE); Egon Haden, Ludwigshafen (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/742,717

(22) PCT Filed: Nov. 14, 2008
(86) PCT No.: PCT/EP2008/065603

§ 371 Date: May 13, 2010

(30) Foreign Application Priority Data
Nov. 15, 2007 (EP) 07120816.9

(51) Int. Cl. A01C 1/06; A61K 31/4427; A61K 31/535

(52) U.S. Cl. 504/100; 514/336; 514/231.2

(57) ABSTRACT

The present invention relates to mixtures of fungicidally active compounds comprising at least one substituted 3-hydroxymethylpyridin compound formula I and at least one further fungicidally active compound II.

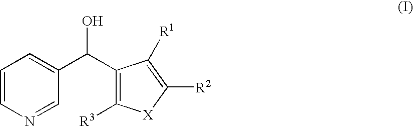

wherein:
X is O, S or NR⁴;
R¹ is C₁-C₆-alkyl, C₁-C₆-alkoxy-C₁-C₆-alkyl, C₁-C₆-haloalkyl, aryl-C₁-C₄-alkyl, aryloxy-C₁-C₄-alkyl, arylthio-C₁-C₄-alkyl, aryl or heteroaryl, wherein the cyclic moieties of the last five radicals are unsubstituted or substituted with 1, 2 or 3 radicals selected from halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro;
R² is H or C₁-C₄-alkyl;
R³ is C₁-C₆-alkyl, C₁-C₆-alkoxy-C₁-C₆-alkyl, C₁-C₆-haloalkyl, aryl-C₁-C₄-alkyl, aryloxy-C₁-C₄-alkyl, arylthio-C₁-C₄-alkyl, aryl or heteroaryl, wherein the cyclic moieties of the last five radicals are unsubstituted or substituted with 1, 2 or 3 radicals selected from halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro;
R⁴ is H, C₁-C₆-alkyl, C₁-C₆-alkoxy-C₁-C₆-alkyl, C₁-C₆-haloalkyl, aryl-C₁-C₄-alkyl, aryloxy-C₁-C₄-alkyl, arylthio-C₁-C₄-alkyl, aryl or heteroaryl, wherein the cyclic moieties of the last five radicals are unsubstituted or substituted with 1, 2 or 3 radicals selected from halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro;

The present invention relates to mixtures of fungicidally active compounds comprising at least one substituted 3-hydroxymethylpyridin compound and at least one further fungicidally active compound.

One typical problem arising in the field of fungicidal control lies in the need to reduce the dosage rates of the active ingredient in order to reduce or avoid unfavorable environmental or toxicological effects whilst still allowing effective pest control.

Another problem encountered concerns the need to have available fungicidal agents which are effective against a broad spectrum of fungi.

There also exists the need for fungicidal agents that combine knock-down activity with prolonged control, that is, fast action with long lasting action.

A further problem arising with the use of fungicides, is that the repeated and exclusive application of an individual fungicidal compound often leads to a rapid selection of harmful fungi which have developed natural or adapted resistance against the active compound in question. Normally, such fungi strains are also cross resistant against other active ingredients having the same mode of action. An effective control of the pathogens with said active compounds is then not possible anymore. However, active ingredients having new mechanisms of action are difficult and expensive to develop.

Another problem underlying the present invention is the desire for compositions that improve plants, a process which is commonly and hereinafter referred to as “plant health”. The term “plant health” comprises various sorts of improvements of plants that are not connected to the control of fungi. For example, advantageous properties that may be mentioned are improved crop characteristics including: emergence, crop yields, protein content, oil content, starch content, more developed root system (improved root growth), improved stress tolerance (e.g. against drought, heat, salt, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination; or any other advantages familiar to a person skilled in the art.

It was therefore an object of the present invention to provide fungicidal mixtures which solve the problems of reducing the dosage rate and/or enhancing the spectrum of activity and/or combining knock-down activity with prolonged control and/or to resistance management and/or promoting the health of plants.

Applicants surprisingly found that these objects are in part or in whole achieved by the combination of certain fungicidal 3-hydroxymethylpyridin compounds of the formula I as defined hereinafter and disclosed in WO 2007/075487 with certain other fungicidally active compounds II.

Especially, it has been found that a mixture of a compound of formula I as defined hereinafter and compound II as defined hereinafter show markedly enhanced action against fungi compared to the control rates that are possible with the individual compounds and/or is suitable for improving the health of plants when applied to plants, parts of plants, seeds, or at their locus of growth.

It has been found that the action of the inventive mixtures goes far beyond the fungicidal and/or plant health improving action of the active compounds present in the mixture alone.

Moreover, we have found that simultaneous, that is joint or separate, application of a compound I and compound II or successive application of a compound I and the compounds II allows enhanced control of harmful fungi, compared to the control rates that are possible with the individual compounds (synergistic mixtures).

Therefore the present invention relates to mixtures of fungicidally active ingredients, comprising, as active components,

1) at least one substituted 3-hydroxymethylpyridin compound of formula I

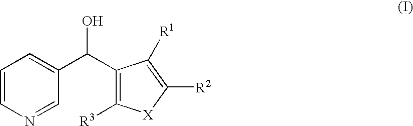

wherein:
X is O, S or NR⁴;
R¹ is C₁-C₆-alkyl, C₁-C₆-alkoxy-C₁-C₆-alkyl, C₁-C₆-haloalkyl, aryl-C₁-C₄-alkyl, aryloxy-C₁-C₄-alkyl, arylthio-C₁-C₄-alkyl, aryl or heteroaryl, wherein the cyclic moieties of the last five radicals are unsubstituted or substituted with 1, 2 or 3 radicals selected from halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro;
R² is H or C₁-C₄-alkyl;
R³ is C₁-C₆-alkyl, C₁-C₆-alkoxy-C₁-C₆-alkyl, C₁-C₆-haloalkyl, aryl-C₁-C₄-alkyl, aryloxy-C₁-C₄-alkyl, arylthio-C₁-C₄-alkyl, aryl or heteroaryl, wherein the cyclic moieties of the last five radicals are unsubstituted or substituted with 1, 2 or 3 radicals selected from halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro;
R⁴ is H, C₁-C₆-alkyl, C₁-C₆-alkoxy-C₁-C₆-alkyl, C₁-C₆-haloalkyl, aryl-C₁-C₄-alkyl, aryloxy-C₁-C₄-alkyl, arylthio-C₁-C₄-alkyl, aryl or heteroaryl, wherein the cyclic moieties of the last five radicals are unsubstituted or substituted with 1, 2 or 3 radicals selected from halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro;
or a salt thereof;
   and
   2) at least one fungicidal compound II selected from:
A) azoles, selected from the group consisting of azaconazole, diniconazole-M, oxpoconazol, uniconazole, 1-(4-chloro-phenyl)-2-([1,2,4]triazol-1-yl)-cycloheptanol, imazalil-sulfphate, bitertanole, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, paclobutrazol, penconazole, propiconazole, prothioconazole, simeconazole, tebuconazole, tetraconazole, uniconazole-P, triadimenol, triadimefon, triticonazole, cyazofamid, imazalil, pefurazoate, prochloraz, triflumizol, benomyl, carbendazim, fuberidazole, thiabendazole, ethaboxam, etridiazole, hymexazole and 1-(4-chloro-phenyl)-1-(propyn-2-yloxy)-3-(4-(3,4-dimethoxy-phenyl)-isoxazol-5-yl)-propane-2-one;
B) strobilurins, selected from the group consisting of 2-(2-(6-(3-chloro-2-methyl-phenoxy)-5-fluoro-pyrimidin-4-yloxy)-phenyl)-2-methoxyimino-N-methyl-acetamide, 3-methoxy-2-(2-(N-(4-methoxy-phenyl)-cyclopropane-carboximidoylsulfanylmethyl)-phenyl)-acrylic acid methyl ester, azoxystrobin, dimoxystrobin, enestroburin, fluoxastrobin, kresoxim-methyl, metominostrobin, orysastrobin, picoxystrobin, pyraclostrobin, trifloxystrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, and methyl 2-(ortho-((2,5-dimethylphenyloxymethylene)phenyl)-3-methoxyacrylate;
C) carboxamides, selected from the group consisting of benalaxyl-M, 2-amino-4-methyl-thiazole-5-carboxylic acid anilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxylic acid amide, N-(4′-chloro-3′,5-difluoro-biphenyl-2-yl)-3-difluoromethyl-1-methyl-1H-pyrazole-4-carboxylic acid amide, N-(4′-chloro-3′,5-difluoro-biphenyl-2-yl)-3-trifluoromethyl-1-methyl-1H-pyrazole-4-carboxylic acid amide, N-(3′,4′-dichloro-5-fluorobiphenyl-2-yl)-3-trifluoromethyl-1-methyl-1H-pyrazole-4-carboxylic acid amide, N-(3′,5-difluoro-4′-methyl-biphenyl-2-yl)-3-difluoromethyl-1-methyl-1H-pyrazole-4-carboxylic acid amide, N-(3′,5-difluoro-4′-methyl-biphenyl-2-yl)-3-trifluoromethyl-1-methyl-1H-pyrazole-4-carboxylic acid amide, N-(cis-2-bicyclopropyl-2-yl-phenyl)-3-difluoromethyl-1-methyl-1H-pyrazole-4-carboxylic acid amide, N-(trans-2-bicyclopropyl-2-yl-phenyl)-3-difluoromethyl-1-methyl-1H-pyrazole-4-carboxylic acid amide, fluopyram, N-(3-ethyl-3,5,5-trimethyl-cyclohexyl)-3-formylamino-2-hydroxy-benzamide, oxytetracyclin, silthiofam, N-(6-methoxy-pyridin-3-yl)cyclopropanecarboxylic acid amide, carboxin, benalaxyl, boscalid, fenhexamid, flutolanil, furametpyr, mepronil, metalaxyl, mefenoxam, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(3′,4′-dichloro-5-fluorobiphenyl-2-yl)-3-difluoromethyl-1-methylpyrazole-4-carboxamide; 3,4-dichloro-N-(2-cyanophenyl)isothiazol-5-carboxamide; N-(2′,4′-difluorobiphenyl-2-yl)-1-methyl-3-trifluoromethyl-1H-pyrazole-4-carboxamide; N-(2′,4′-dichlorobiphenyl-2-yl)-1-methyl-3-trifluoromethyl-1H-pyrazole-4-carboxamide; N-(2′,4′-difluorobiphenyl-2-yl)-3-difluoromethyl-1-methyl-1H-pyrazole-4-carboxamide; N-(2′,4′-dichlorobiphenyl-2-yl)-3-difluoromethyl-1-methyl-1H-pyrazole-4-carboxamide; N-(2′,5′-difluorobiphenyl-2-yl)-1-methyl-3-trifluoromethyl-1H-pyrazole-4-carboxamide; N-(2′,5′-dichlorobiphenyl-2-yl)-1-methyl-3-trifluoromethyl-1H-pyrazole-4-carboxamide; N-(2′,5′-difluorobiphenyl-2-yl)-3-difluoromethyl-1-methyl-1H-pyrazole-4-carboxamide; N-(2′,5′-dichlorobiphenyl-2-yl)-3-difluoromethyl-1-methyl-1H-pyrazole-4-carboxamide; N-(3′,5′-difluorobiphenyl-2-yl)-1-methyl-3-trifluoromethyl-1H-pyrazole-4-carboxamide; N-(3′,5′-dichlorobiphenyl-2-yl)-1-methyl-3-trifluoromethyl-1H-pyrazole-4-carboxamide, N-(3′,5′-difluorobiphenyl-2-yl)-3-difluoromethyl-1-methyl-1H-pyrazole-4-carboxamide; N-(3′,5′-dichlorobiphenyl-2-yl)-3-difluoromethyl-1-methyl-1H-pyrazole-4-carboxamide; N-(3′-fluorobiphenyl-2-yl)-1-methyl-3-trifluoromethyl-1H-pyrazole-4-carboxamide, N-(3′-chlorobiphenyl-2-yl)-1-methyl-3-trifluoromethyl-1H-pyrazole-4-carboxamide; N-(3′-fluorobiphenyl-2-yl)-3-difluoromethyl-1-methyl-1H-pyrazole-4-carboxamide, N-(3′-chlorobiphenyl-2-yl)-3-difluoromethyl-1-methyl-1H-pyrazole-4-carboxamide; N-(2′-fluorobiphenyl-2-yl)-1-methyl-3-trifluoromethyl-1H-pyrazole-4-carboxamide; N-(2′-chlorobiphenyl-2-yl)-1-methyl-3-trifluoromethyl-1H-pyrazole-4-carboxamide; N-(2′-fluorobiphenyl-2-yl)-3-difluoromethyl-1-methyl-1H-pyrazole-4-carboxamide; N-(2′-chlorbiphenyl-2-yl)-3-difluoromethyl-1-methyl-1H-pyrazole-4-carboxamide; N-(2′-fluoro-4′-chloro-5′-methylbiphenyl-2-yl)-1-methyl-3-trifluoromethyl-1H-pyrazole-4-carboxamide; N-(3′,4′,5′-trifluorobiphenyl-2-yl)-1-methyl-3-trifluoromethyl-1H-pyrazole-4-carboxamide; N-(3′,4′,5′-trifluorobiphenyl-2-yl)-1-methyl-3-difluoromethyl-1H-pyrazole-4-carboxamide; N-(2′,4′,5′-trifluorobiphenyl-2-yl)-1-methyl-3-difluoromethyl-1H-pyrazole-4-carboxamide; N-(3′,4′,5′-trifluorobiphenyl-2-yl)-3-chlorofluoromethyl-1-methyl-1H-pyrazole-4-carboxamide; N-[2-(1,1,2,3,3,3-hexafluoropropoxy)phenyl]-1-methyl-3-trifluoromethyl-1H-pyrazole-4-carboxamide; N-[2-(1,1,2,3,3,3-hexafluoropropoxy)-phenyl]-3-difluoromethyl-1-methyl-1H-pyrazole-4-carboxamide, N-[2-(2-chloro-1,1,2-trifluoroethoxy)phenyl]-1-methyl-3-trifluoromethyl-1H-pyrazole-4-carboxamide; N-[2-(2-chlor-1,1,2-trifluoroethoxy)phenyl]-3-difluoromethyl-1-methyl-1H-pyrazole-4-carboxamide; N-[2-(1,1,2,2-tetrafluoroethoxy)phenyl]-3-difluoromethyl-1-methyl-1H-pyrazole-4-carboxamide; N-[2-(1,1,2,2-tetrafluoroethoxy)phenyl]-1-methyl-3-trifluoromethyl-1H-pyrazole-4-carboxamide; N-(4′-(trifluoromethylthio)biphenyl-2-yl)-3-difluoromethyl-1-methyl-1H-pyrazole-4-carboxamide; N-(4′-(trifluoromethylthio)biphenyl-2-yl)-1-methyl-3-trifluoromethyl-1H-pyrazole-4-carboxamide; dimethomorph, flumorph;
   flumetover, fluopicolide (picobenzamid), zoxamide;
   carpropamid, diclocymet, mandipropamid; N-(2-{4-[3-(4-chlorophenyl)prop-2-ynyloxy]-3-methoxyphenyl}ethyl)-2-methanesulfonylamino-3-methylbutyramide, N-(2-{4-[3-(4-chlorophenyl)-prop-2-ynyloxy]-3-methoxyphenyl}ethyl)-2-ethanesulfonylamino-3-methylbutyramide;
D) heterocyclic compounds, selected from the group consisting of 2,3,5,6-tetra-chloro-4-methanesulfonyl-pyridine, 3,4,5-trichloropyridine-2,6-dicarbonitrile, N-(1-(5-bromo-3-chloro-pyridin-2-yl)-ethyl)-2,4-dichloronicotinamide, N-[(5-bromo-3-chloro-pyridin-2-yl)-methyl]-2,4-dichloro-nicotinamide, diflumetorim, nitrapyrin, dodemorph acetate, fluoroimid, blasticidin-S, chinomethionat, debacarb, difenzoquat, difenzoquat-methylsulphat, oxolinic acid, piperalin, fluazinam, pyrifenox; bupirimate, cyprodinil, fenarimol, ferimzone, mepanipyrim, nuarimol, pyrimethanil;
   triforine;
   fenpiclonil, fludioxonil;
   aldimorph, dodemorph, fenpropimorph, tridemorph; fenpropidin, iprodione, procymidone, vinclozolin;
   famoxadone, fenamidone, octhilinone, probenazole;
   amisulbrom, anilazine, diclomezine, pyroquilon, proquinazid, tricyclazole;
   2-butoxy-6-iodo-3-propylchromen-4-one;
   acibenzolar-S-methyl, captafol, captan, dazomet, folpet, fenoxanil, quinoxyfen; 3-[5-(4-chlorophenyl)-2,3-dimethylisoxazolidin-3-yl]pyridine, 5-chloro-7-(4-methylpiperidin-1-yl)-6-(2,4,6-trifluorophenyl)-[1,2,4]triazolo[1,5-a]pyrimidine, 6-(3,4-dichloro-phenyl)-5-methyl-[1,2,4]triazolo[1,5-a]pyrimidine-7-ylamine, 6-(4-tert-butylphenyl)-5-methyl-[1,2,4]triazolo[1,5-a]pyrimidine-7-ylamine, 5-methyl-6-(3,5,5-trimethyl-hexyl)-[1,2,4]triazolo[1,5-a]pyrimidine-7-ylamine, 5-methyl-6-octyl-[1,2,4]triazolo[1,5-a]pyrimidine-7-ylamine, 6-methyl-5-octyl-[1,2,4]triazolo[1,5-a]pyrimidine-7-ylamine, 6-ethyl-5-octyl-[1,2,4]triazolo[1,5-a]pyrimidine-7-ylamine, 5-ethyl-6-octyl-[1,2,4]triazolo[1,5-a]pyrimidine-7-ylamine, 5-ethyl-6-(3,5,5-trimethyl-hexyl)-[1,2,4]triazolo[1,5-a]pyrimidine-7-ylamine, 6-octyl-5-propyl-[1,2,4]triazolo[1,5-a]pyrimidine-7-ylamine, 5-methoxymethyl-6-octyl-[1,2,4]triazolo[1,5-a]pyrimidine-7-ylamine, 6-octyl-5-trifluoromethyl-[1,2,4]triazolo[1,5-a]pyrimidine-7-ylamine and 5-trifluoromethyl-6-(3,5,5-trimethyl-hexyl)-[1,2,4]triazolo[1,5-a]pyrimidine-7-ylamine;
E) carbamates, selected from the group consisting of methasulphocarb, propamocarb hydrochlorid, mancozeb, maneb, metam, metiram, ferbam, propineb, thiram, zineb, ziram, thiophanate-methyl;
   diethofencarb, iprovalicarb, flubenthiavalicarb, propamocarb;
   methyl 3-(4-chlorophenyl)-3-(2-isopropoxycarbonylamino-3-methyl-butyrylamino)propanoate, 4-fluorophenyl N-(1-(1-(4-cyanophenyl)ethanesulfonyl)-but-2-yl)carbamate;
and
F) other active compounds, selected from the group consisting of guanidines: dodine, iminoctadine, guazatine, dodine free base, guazatine-acetate, iminoctadine-triacetate;
   antibiotics: kasugamycin, streptomycin, polyoxine, validamycin A;
   nitrophenyl derivates: binapacryl, dinocap, dinobuton;
   sulfur-containing heterocyclyl compounds: dithianon, isoprothiolane;
   organometal compounds: fentin salts, such as fentin acetate;
   organophosphorus compounds: edifenphos, iprobenfos, fosetyl, fosetyl-aluminum, phosphorous acid and its salts, pyrazophos, toiclofos-methyl;
   organochlorine compounds: chlorothalonil, dichlofluanid, flusulfamide, hexachlorobenzene, phthalide, pencycuron, quintozene, tolylfluanid;
   inorganic active compounds: Bordeaux mixture, copper acetate, copper hydroxide, copper oxychloride, basic copper sulfate, sulfur;
   others: cyflufenamid, cymoxanil, dimethirimol, ethirimol, furalaxyl, metrafenone, spiroxamine, iminoctadine-tris(albesilate), kasugamycin-hydrochlorid-hydrat, dichlorophen, pentachlorophenol and its salts, N-(4-chloro-2-nitro-phenyl)-N-ethyl-4-methyl-benzenesulfonamide, dicloran, nitrothal-isopropyl, tecnazen, biphenyl, bronopol, diphenylamine, mildiomycin, oxin-copper, prohexadione calcium, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine and N′-(5-difluoromethyl-2-methyl-4-(3-trimethylsilanyl-propoxy)-phenyl)-N-ethyl-N-methyl formamidine;
      in synergistically effective relative amounts.

This invention also relates to a method for controlling harmful fungi using the inventive mixtures and to the use of a compound I and the active compound II for preparing such mixtures, and also to compositions comprising such mixtures.

The present invention further relates to plant-protecting active ingredient mixtures having synergistically enhanced action of improving the health of plants and to a method of applying such inventive mixtures to the plants.

The compounds described herein and, optionally, all their isomers may be used in the form of their salts. Because some of the compounds I have a basic center they can, for example, form acid addition salts. Said acid addition salts are, for example, formed with mineral acids, typically sulfuric acid, a phosphoric acid or a hydrogen halide, with organic carboxylic acids, typically acetic acid, oxalic acid, malonic acid, maleic acid, fumaric acid or phthalic acid, with hydroxycarboxylic acids, typically ascorbic acid, lactic acid, malic acid, tartaric acid or citric acid, or with benzoic acid, or with organic sulfonic acids, typically methanesulfonic acid or p-toluenesulfonic acid. Together with at least one acidic group, the compounds of formula I can also form salts with bases. Suitable salts with bases are, for example, metal salts, typically alkali metal salts; or alkaline earth metal salts, e.g. sodium salts, potassium salts or magnesium salts, or salts with ammonia or an organic amine, e.g. morpholine, piperidine, pyrrolidine, a mono-, di- or trialkylamine, typically ethylamine, diethylamine, triethylamine or dimethylpropylamine, or a mono-, di- or trihydroxyalkylamine, typically mono-, di- or triethanolamine. Where appropriate, the formation of corresponding internal salts is also possible. Within the scope of this invention, agrochemical acceptable salts are preferred.

The term Cₙ—Cₘ indicates the number of carbon atoms of an individual substituent or the respective part of a substituent.

“Halo” or “halogen”, as used herein, refers to Cl, Br, I or F.

“Cyano” as used herein refers to a CN group.

“Alkyl” as used herein refers to a saturated hydrocarbon radical which may be straight-chain or branched-chain (for example, ethyl, isopropyl, t-amyl, or hexyl) and contains from 1 to 6, in particular 1 to 4 carbon atoms. This definition applies both when the term is used alone and when it is used as part of a compound term, such as “haloalkyl”, “alkoxy”, “alkoxyalkyl”, “alkylthio” and similar terms. In some embodiments, preferred alkyl groups are those containing 1 to 4 carbon atoms.

“Alkylene” as used herein refers to a bivalent saturated hydrocarbon radical which may be straight-chain or branched-chain (for methylene (CH₂), 1,1-ethandiyl, 1,2-ethandiyl, 1,1-propandiyl, 1,2-propandiyl, 1,3-propandiyl, 1,1-butandiyl, 1,2-butandiyl, 1,3-butandiyl, 2,3-butandiyl or 1,4-butandiyl.

“Haloalkyl”, as used herein, refers to alkyl, as defined herein, wherein the hydrogen atoms are partly or completely replaced by halogen atoms, in particular fluorine or chlorine atoms. Representative examples of haloalkyl include, but are not limited to, chloromethyl, 2-fluoroethyl, fluoromethyl, difluoromethyl, trifluoromethyl, 2,2,2-trifluoroethyl, pentafluoroethyl, and the like. This definition applies both when the term is used alone and when it is used as part of a compound term, such as “haloalkyloxy”, “haloalkylthio” and similar terms. In some embodiments, preferred haloalkyl groups are those containing 1 or 2 carbon atoms.

“Alkenyl” as used herein, refers to a straight or branched chain hydrocarbon containing from 2 to 6 carbons and containing at least one carbon-carbon double bond formed by the removal of two hydrogens. Representative examples of “alkenyl” include, but are not limited to, ethenyl, 2-propenyl, 2-methyl-2-propenyl, 3-butenyl, 4-pentenyl, 5-hexenyl, and the like. “Lower alkenyl” as used herein, is a subset of alkenyl and refers to a straight or branched chain hydrocarbon group containing from 2 to 4 carbon atoms.

“Alkynyl” as used herein, refers to a straight or branched chain hydrocarbon group containing from 2 to 6 carbon atoms and containing at least one carbon-carbon triple bond. Representative examples of alkynyl include, but are not limited, to ethynyl, 1-propynyl, 2-propynyl, 3-butynyl, 2-pentynyl, 1-butynyl and the like. “Lower alkynyl” as used herein, is a subset of alkynyl and refers to a straight or branched chain hydrocarbon group containing from 2 to 4 carbon atoms.

“Alkoxy” refers to an alkyl radical as described above which is bound to the remainder of the molecule via an oxygen atom (such as, for example, methoxy, ethoxy and t-butoxy).

“Alkylthio” as used herein refers to an alkyl group, as defined herein, appended to the parent molecular moiety through a sulfur atom. Representative examples of alkylthio include, but are not limited, methylthio, ethylthio, tert-butylthio, and the like.

“Haloalkoxy” refers to an haloalkyl radical as described above which is bound to the remainder of the molecule via an oxygen atom (such as, for example, 2-fluoroethoxy, fluoromethoxy, difluoromethoxyl, trifluoromethoxy, 2,2,2-trifluoroethoxy, pentafluoroethoxy and the like).

“Haloalkylthio” as used herein refers to a haloalkyl group, as defined herein, appended to the parent molecular moiety through a sulfur atom. Representative examples of alkylthio include, but are not limited to 2-fluoroethylthio, fluoromethylthio, difluoromethylthio, trifluoromethylthio, 2,2,2-trifluoroethylthio, pentafluoroethylthio and the like.

“Aryl” or “aromatic ring moiety” refers to an aromatic hydrocarbon substituent which may be a single ring or multiple rings which are fused together, linked covalently or linked to a common group such as an ethylene or methylene moiety. Representative examples of aryl include, azulenyl, indanyl, indenyl, naphthyl, phenyl, tetrahydronaphthyl, biphenyl, diphenylmethyl, 2,2-diphenyl-1-ethyl, in particular phenyl.

“Heteroaryl” or “Hetaryl” means a cyclic, aromatic radical in which one or more, e.g. 1, 2, 3 or 4, ring carbon atoms have been replaced by heteroatoms such as O, S or N. If the heteroaryl group contains more than one heteroatom, the heteroatoms may be the same or different. Examples of heteroaryl groups include pyridyl, pyrimidinyl, imidazolyl, thienyl, furyl, pyrrolyl, pyranyl, benzofuranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, benzothiazolyl, pyrido[5,4-b]thiazolyl and benzo[b]thienyl. Preferred heteroaryl groups are five and six membered rings and contain from one to three heteroatoms independently selected from O, N, and S.

“Arylalkyl” refers to an aromatic hydrocarbon substituent as defined above which is linked to the remainder of the molecule by alkylene moiety such as an 1,1-, 1,3- or 1,2-propylene, 1,1- or 1,2-ethylene or methylene moiety. Representative examples of aryl-alkyl include benzyl, 1-phenylethyl, 2-phenylethyl, 1-phenylpropyl, 2-phenylpropyl and 1-methyl-2-phenylethyl.

“Hetarylalkyl” or “heteroarylalkyl” refers to a hetaryl radical as defined above which is linked to the remainder of the molecule by alkylene moiety such as an 1,1-, 1,3- or 1,2-propylene, 1,1- or 1,2-ethylene or methylene moiety. Representative examples of hetarylalkyl include hetarylmethyl, 1-hetarylethyl, 2-hetarylethyl, 1-hetarylpropyl, 2-hetarylpropyl and 1-methyl-2-hetarylethyl.

“Aryloxyalkyl” refers to an aromatic hydrocarbon substituent as defined above which is linked to the remainder of the molecule by O-alkylene moiety such as an O—CH₂, O—CH(CH₃), O—CH₂CH₂, O—CH(CH₃)CH₂ or O—CH₂—CH(CH₃)-moiety. Representative examples of aryloxyalkyl include phenoxymethyl, 1-phenoxyethyl, 2-phenoxyethyl, 1-phenoxypropyl, 2-phenoxypropyl and 1-methyl-2-phenoxyethyl.

“Arylthioalkyl” refers to an aromatic hydrocarbon substituent as defined above which is linked to the remainder of the molecule by S-alkylene moiety such as an S—CH₂, S—CH(CH₃), S—CH₂CH₂, S—CH(CH₃)CH₂ or S—CH₂—CH(CH₃)-moiety. Representative examples of arylthioalkyl include phenylthiomethyl, 1-phenylthioethyl, 2-phenylthioethyl, 1-phenylthiopropyl, 2-phenylthiopropyl and 1-methyl-2-phenylthioethyl.

“Agriculturally acceptable salt” means a salt the cation of which is known and accepted in the art for the formation of salts for agricultural or horticultural use. Preferably the salts are water-soluble.

Preferred compounds of formula I are those, wherein the variables R¹, R² and R³, each independently, or preferably in combination have one of the following meanings:
R¹ phenyl or 5- to 6-membered hetaryl having 1 or 2 heteroatoms selected from O, S and N, in particular phenyl, thienyl or furyl, wherein the cyclic moieties of the radicals are unsubstituted or substituted with 1, 2 or 3 radicals selected from halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro, in particular selected from halogen, C₁-C₂-alkyl or C₁-C₂-haloalkyl. Particularly preferred examples of R¹ include 2-chlorophenyl, 3-chlorophenyl, 4-chlorophenyl, 2,4-dichlorophenyl, 2-chloro-4-fluorophenyl, 2-fluorophenyl, 3-fluorophenyl, 4-fluorophenyl, 2,4-difluorophenyl, 2,5-difluorophenyl, 3,5-difluorophenyl, 3-chloro-4-fluorophenyl, 4-chloro-2-fluorophenyl, 2-chloro-5-trifluoromethylphenyl, 2-chloro-4-trifluoromethylphenyl, 2-trifluoromethylphenyl, 3-trifluoromethylphenyl, 4-trifluoromethylphenyl, 4-methoxyphenyl, 4-trifluoromethoxyphenyl, 2-thienyl, 5-chloro-2-thienyl, 5-methyl-2-thienyl, 3-thienyl, 2-furyl and 5-chloro-2-furyl.
R² hydrogen;
R³ phenyl or 5- to 6-membered hetaryl having 1 or 2 heteroatoms selected from O, S and N, in particular phenyl or thienyl, wherein the cyclic moieties of the radicals are unsubstituted or substituted with 1, 2 or 3 radicals selected from halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro, in particular selected from halogen, C₁-C₂-alkyl or C₁-C₂-haloalkyl. Particularly preferred examples of R³ include 2-chlorophenyl, 3-chlorophenyl, 4-chlorophenyl, 2,4-dichlorophenyl, 2-chloro-4-fluorophenyl, 2-fluorophenyl, 3-fluorophenyl, 4-fluorophenyl, 2,4-difluorophenyl, 2,5-difluorophenyl, 3,5-difluorophenyl, 3-chloro-4-fluorophenyl, 4-chloro-2-fluorophenyl, 2-chloro-5-trifluoromethylphenyl, 2-chloro-4-trifluoromethylphenyl, 2-trifluoromethylphenyl, 3-trifluoromethylphenyl, 4-trifluoromethylphenyl, 4-methoxyphenyl, 4-trifluoromethoxyphenyl, 2-thienyl, 5-chloro-2-thienyl, 5-bromo-2-thienyl and 5-methyl-2-thienyl and 3-thienyl.

A first embodiment of the invention relates to mixtures, comprising a compound of the formula I, wherein X is S, and wherein R¹, R² and R³ are as defined herein and in particular have one of the preferred or particularly preferred or exemplified meanings.

A second embodiment of the invention relates to mixtures, comprising a compound of the formula I, wherein X is O, and wherein R¹, R² and R³ are as defined herein and in particular have one of the preferred or particularly preferred or exemplified meanings.

A third embodiment of the invention relates to mixtures, comprising a compound of the formula I, wherein X is N—R⁴, wherein R⁴ is as defined above, and wherein R¹, R² and R³ are as defined herein and in particular have one of the preferred or particularly preferred or exemplified meanings. In this embodiment, R⁴ is preferably C₁-C₄-alkyl and especially methyl.

Preferred compounds of formula I are those, wherein:
R¹ is phenyl or 5- to 6-membered hetaryl having 1 or 2 heteroatoms selected from O, S and N, in particular phenyl, thienyl or furyl, wherein the cyclic moieties of the radicals are unsubstituted or substituted with 1, 2 or 3 radicals selected from halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro, in particular selected from halogen, C₁-C₂-alkyl or C₁-C₂-haloalkyl. Particularly preferred examples of R¹ include 2-chlorophenyl, 3-chlorophenyl, 4-chlorophenyl, 2,4-dichlorophenyl, 2-chloro-4-fluorophenyl, 2-fluorophenyl, 3-fluorophenyl, 4-fluorophenyl, 2,4-difluorophenyl, 2,5-difluorophenyl, 3,5-difluorophenyl, 3-chloro-4-fluorophenyl, 4-chloro-2-fluorophenyl, 2-chloro-5-trifluoromethylphenyl, 2-chloro-4-trifluoromethylphenyl, 2-trifluoromethylphenyl, 3-trifluoromethylphenyl, 4-trifluoromethylphenyl, 4-methoxyphenyl, 4-trifluoromethoxyphenyl, 2-thienyl, 5-chloro-2-thienyl, 5-methyl-2-thienyl, 3-thienyl, 2-furyl and 5-chloro-2-furyl.
R² hydrogen;
R³ is phenyl or 5- to 6-membered hetaryl having 1 or 2 heteroatoms selected from O, S and N, in particular phenyl or thienyl, wherein the cyclic moieties of the radicals are unsubstituted or substituted with 1, 2 or 3 radicals selected from halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro, in particular selected from halogen, C₁-C₂-alkyl or C₁-C₂-haloalkyl. Particularly preferred examples of R³ include 2-chlorophenyl, 3-chlorophenyl, 4-chlorophenyl, 2,4-dichlorophenyl, 2-chloro-4-fluorophenyl, 2-fluorophenyl, 3-fluorophenyl, 4-fluorophenyl, 2,4-difluorophenyl, 2,5-difluorophenyl, 3,5-difluorophenyl, 3-chloro-4-fluorophenyl, 4-chloro-2-fluorophenyl, 2-chloro-5-trifluoromethylphenyl, 2-chloro-4-trifluoromethylphenyl, 2-trifluoromethylphenyl, 3-trifluoromethylphenyl, 4-trifluoromethylphenyl, 4-methoxyphenyl, 4-trifluoromethoxyphenyl, 2-thienyl, 5-chloro-2-thienyl, 5-bromo-2-thienyl and 5-methyl-2-thienyl and 3-thienyl;
   and
X is S.

Examples of compounds of the present invention include, but are not limited to, the following compounds 1.1 to 1.61, listed in table A:

TABLE A

| | |
|---|---|
| I.1 | 2,4-Bis-(3-chlorophenyl)-3-[(3-pyridyl)hydroxymethyl]thiophene; |
| I.2 | 4-(3-Chlorophenyl)-2-(5-chloro-2-thienyl)-3-[(3-pyridyl)hydroxymethyl]thiophene; |
| I.3 | 4-(3-Chlorophenyl)-2-(3,5-difluorophenyl)-3-[(3-pyridyl)hydroxymethyl]thiophene; |
| I.4 | 4-(4-Chlorophenyl)-2-(5-chloro-2-thienyl)-3-[(3-pyridyl)hydroxymethyl]thiophene; |
| I.5 | 4-(4-Chlorophenyl)-2-(3,5-difluorophenyl)-3-[(3-pyridyl)hydroxymethyl]thiophene; |
| I.6 | 2-(4-Chlorophenyl)-4-(2,4-difluorophenyl)-3-[(3-pyridyl)hydroxymethyl]thiophene; |
| I.7 | 4-(2,4-Difluorophenyl)-2-(1,1-dimethylethyl)-3-[(3- |
| | pyridyl)hydroxymethyl]thiophene |
| I.8 | 2,4-Bis-(4-chlorophenyl)-3-[(3-pyridyl)hydroxymethyl]thiophene; |
| I.9 | 4-(4-Chlorophenyl)-3-[(3-pyridyl)hydroxymethyl]-2-(2-thienyl)thiophene; |
| I.10 | 2-(4-Chlorophenyl)-4-(5-chloro-2-thienyl)-3-[(3-pyridyl)hydroxymethyl]thiophene; |
| I.11 | 4-(5-Chloro-2-thienyl)-2-(2,4-difluorophenyl)-3-[(3-pyridyl)hydroxymethyl]- |
| | thiophene; |
| I.12 | 2-(4-Chlorophenyl)-3-[(3-pyridyl)hydroxymethyl]-4-(2-thienyl)thiophene |
| I.13 | 2-(2,4-Difluorophenyl)-3-[(3-pyridyl)hydroxymethyl]-4-(2-thienyl)thiophene; |
| I.14 | 2-(2,4-Difluorophenyl)-3-[(3-pyridyl)hydroxymethyl]-4-(2-thienyl)thiophene; |
| I.15 | 2-(4-Butylphenyl)-4-(5-methyl-2-thienyl)-3-[(3-pyridyl)hydroxymethyl]thiophene; |
| I.16 | 2,4-Bis-(2,4-Difluorophenyl)-3-[(3-pyridyl)hydroxymethyl]thiophene |
| I.17 | 4-(4-Chlorophenyl)-2-(2,4-difluorophenyl)-3-[(3-pyridyl)hydroxymethyl]thiophene; |
| I.18 | 2,4-Bis-(2-trifluoromethylphenyl)-3-[(3-pyridyl)hydroxymethyl]thiophene; |
| I.19 | 2,4-Bis-(3-trifluoromethylphenyl)-3-[(3-pyridyl)hydroxymethyl]thiophene; |
| I.20 | 2,4-Bis-(4-trifluoromethylphenyl)-3-[(3-pyridyl)hydroxymethyl]thiophene; |
| I.21 | 4-(4-Chlorophenyl)-3-[(3-pyridyl)hydroxymethyl]-2-(3-thienyl)thiophene; |
| I.22 | 2-(5-Bromo-2-thienyl)-4-(4-chlorophenyl)-3-[(3-pyridyl)hydroxymethyl]thiophene; |
| I.23 | 4-(4-Chlorophenyl)-2-(5-methyl-2-thienyl)-3-[(3-pyridyl)hydroxymethyl]thiophene; |
| I.24 | 2-(3,5-Difluorophenyl)-3-[(3-pyridyl)hydroxymethyl]-4-(3-thienyl)thiophene; |
| I.25 | 2-(2,4-Difluorophenyl)-3-[(3-pyridyl)hydroxymethyl]-4-(3-thienyl)thiophene; |
| I.26 | 2-(3,5-Difluorophenyl)-4-(4-fluorophenyl)-3-[(3-pyridyl)hydroxymethyl]thiophene; |
| I.27 | 2-(2,4-Difluorophenyl)-4-(4-fluorophenyl)-3-[(3-pyridyl)hydroxymethyl]thiophene; |
| I.28 | 2-(4-Chlorophenyl)-3-[(3-pyridyl)hydroxymethyl]-4-(3-thienyl)thiophene; |
| I.29 | 4-(2,4-Difluorophenyl)-3-[(3-pyridyl)hydroxymethyl]-2-(3-thienyl)thiophene; |
| I.30 | 4-(2,4-Difluorophenyl)-3-[(3-pyridyl)hydroxymethyl]-2-(2-thienyl)thiophene; |
| I.31 | 2-(2,4-Difluorophenyl)-4-(2-fluorophenyl)-3-[(3-pyridyl)hydroxymethyl]thiophene; |
| I.32 | 2,4-Bis-(2-chlorophenyl)-3-[(3-pyridyl)hydroxymethyl]-thiophene; |
| I.33 | 2,4-Bis-(3-chlorophenyl)-3-[(3-pyridyl)hydroxymethyl]thiophene; |
| I.34 | 2,4-Bis-(phenyl)-3-[(3-pyridyl)hydroxymethyl]thiophene; |
| I.35 | 2,4-Bis-(2,4-Dichlorophenyl)-3-[(3-pyridyl)hydroxymethyl]thiophene; |
| I.36 | 2,4-Bis-(2-fluorophenyl)-3-[(3-pyridyl)hydroxymethyl]thiophene; |
| I.37 | 2,4-Bis-(3-fluorophenyl)-3-[(3-pyridyl)hydroxymethyl]thiophene; |
| I.38 | 2-(3-Chlorophenyl)-4,5-dimethyl-3-[(3-pyridyl)hydroxymethyl]thiophene; |
| I.39 | 4-(5-Chloro-2-furanyl)-2-(4-chlorophenyl)-3-[(3-pyridyl)hydroxymethyl]thiophene; |
| I.40 | 4-(5-Chloro-2-furanyl)-2-(2,4-difluorophenyl)-3-[(3-pyridyl)hydroxymethyl]- |
| | thiophene; |
| I.41 | 2,4-Bis-(2-thienyl)-3-[(3-pyridyl)hydroxymethyl]thiophene; |
| I.42 | 2,4-Bis-(4-fluorophenyl)-3-[(3-pyridyl)hydroxymethyl]thiophene; |
| I.43 | 2-(3-Chlorophenyl)-4-phenyl-3-[(3-pyridyl)hydroxymetliyl]thiophene; |
| I.44 | 2,4-Bis-(3-chloro-5-trifluorormethylphenyl)-3-[(3-pyridyl)hydroxymethyl]- |
| | thiophene; |
| I.45 | 2,4-Bis-(2,5-difluorophenyl)-3-[(3-pyridyl)hydroxymethyl]thiophene; |
| I.46 | 2,4-Bis-(4-chloro-3-fluorophenyl)-3-[(3-pyridyl)hydroxymethyl]thiophene; |
| I.47 | 2,4-Bis-(3-methoxyphenyl)-3-[(3-pyridyl)hydroxymethyl]-thiophene; |
| I.48 | 4-(2-Fluorophenyl)-3-[(3-pyridyl)hydroxymethyl]-2-(2-thienyl)thiophene; |
| I.49 | 2,4-Bis-(2-chloro-4-trifluoromethylphenyl)-3-[(3-pyridyl)hydroxymethyl]thiophene; |
| I.50 | 2,4-Bis-(4-methoxyphenyl)-3-[(3-pyridyl)hydroxymethyl]thiophene; |
| I.51 | 2-(3-Chlorophenyl)-4-(2,4-difluorophenyl)-3-[(3-pyridyl)hydroxymethyl]-thiophene; |
| I.52 | 2-(5-Bromo-2-thienyl)-4-(2,4-difluorophenyl)-3-[(3-pyridyl)hydroxymethyl]- |
| | thiophene; |
| I.53 | 2-(5-Chloro-2-thienyl)-4-(2,4-difluorophenyl)-3-[(3-pyridyl)hydroxymethyl]- |
| | thiophene; |
| I.54 | 5-Chloro-2-(5-chloro-2-thienyl)-4-(2,4-difluorophenyl)-3-[(3- |
| | pyridyl)hydroxymethyl]-thiophene; |
| I.55 | 4-(4-Chlorophenyl)-2-(2-fluorophenyl)-3-[(3-pyridyl)hydroxymethyl]thiophene; |
| I.56 | 4-(4-Chlorophenyl)-2-(3-fluorophenyl)-3-[(3-pyridyl)hydroxymethyl]thiophene; |
| I.57 | 2-(2-Chlorophenyl)-4-(2,4-difluorophenyl)-3-[(3-pyridyl)hydroxymethyl]thiophene; |
| I.58 | 4-(2,4-Difluorophenyl)-2-(2-fluorophenyl)-3-[(3-pyridyl)hydroxymethyl]thiophene; |
| I.59 | 2-(4-Chlorophenyl)-4-(4-chloro-2-fluorophenyl)-3-[(3-pyridyl)hydroxymethyl]- |
| | thiophene; |
| I.60 | 2-(3-Chlorophenyl)-4-(4-chloro-2-fluorophenyl)-3-[(3-pyridyl)hydroxymethyl]- |
| | thiophene; and |
| I.61 | 4-(2,4-Difluorophenyl)-2-(4-fluorophenyl)-3-[(3-pyridyl)hydroxymethyl]thiophene; |

and salts thereof.

Particular preference is given to mixtures wherein the compound I is selected from the compounds 1.8 and 1.16.

The active compounds II mentioned above, their preparation and their action against harmful fungi are generally known (cf.: http://www.hclrss.demon.co.uk/index.html); they are commercially available. Particularly, they are known from: bitertanol, 8-([1,1′-biphenyl]-4-yloxy)-α-(1,1-dimethylethyl)-1H-1,2,4-triazole-1-ethanol (DE 23 24 020), bromuconazole, 1-[[4-bromo-2-(2,4-dichlorophenyl)tetrahydro-2-furanyl]methyl]-1H-1,2,4-triazole (Proc. 1990 Br. Crop. Prot. Conf.—Pests Dis. Vol. 1, p. 459); cyproconazole, 2-(4-chlorophenyl)-3-cyclopropyl-1-[1,2,4]triazol-1-ylbutan-2-ol (U.S. Pat. No. 4,664,696); difenoconazole, 1-{2-[2-chloro-4-(4-chlorophenoxy)phenyl]-4-methyl-[1,3]dioxolan-2-ylmethyl}-1H-[1,2,4]triazole (GB-A 2 098 607); diniconazole, (βE)-β-[(2,4-dichlorophenyl)methylene]-α-(1,1-dimethylethyl)-1H-1,2,4-triazole-1-ethanol (Noyaku Kagaku, 1983, Vol. 8, p. 575); enilconazole (imazalil), 1-[2-(2,4-dichlorophenyl)-2-(2-propenyloxy)ethyl]-1H-imidazole (Fruits, 1973, Vol. 28, p. 545); epoxiconazole, (2RS,3SR)-1-[3-(2-chlorophenyl)-2,3-epoxy-2-(4-fluorophenyl)propyl]-1H-1,2,4-triazole (EP-A 196 038); fluquinconazole, 3-(2,4-dichlorophenyl)-6-fluoro-2-[1,2,4]-triazol-1-yl-3H-quinazolin-4-one (Proc. Br. Crop Prot. Conf.—Pests Dis., 5-3, 411 (1992)); fenbuconazole, α-[2-(4-chlorophenyl)ethyl]-α-phenyl-1H-1,2,4-triazole-1-propanenitrile (Proc. 1988 Br. Crop Prot. Conf.—Pests Dis. Vol. 1, p. 33); flusilazole, 1-{[bis-(4-fluorophenyl)methylsilanyl]methyl}-1H-[1,2,4]triazole (Proc. Br. Crop Prot. Conf.—Pests Dis., 1, 413 (1984)); flutriafol, α-(2-fluorophenyl)-α-(4-fluorophenyl)-1H-1,2,4-triazole-1-ethanol (EP 15 756); hexaconazole, 2-(2,4-dichlorophenyl)-1-[1,2,4]triazol-1-ylhexan-2-ol (CAS RN 79983-71-4); imibenconazole, (4-chlorophenyl)methyl N-(2,4-dichlorophenyl)-1H-1,2,4-triazole-1-ethanimidothioate ((Proc. 1988 Br. Crop Prot. Conf.—Pests Dis. Vol. 2, p. 519); imidothioate ((Proc. 1988 Br. Crop Prot. Conf.—Pests Dis. Vol. 2, p. 519); ipconazole, 2-[(4-chlorophenyl)methyl]-5-(1-methylethyl)-1-(1H-1,2,4-triazol-1-ylmethyl)cyclopentanol (EP 267 778); metconazole, 5-(4-chlorobenzyl)-2,2-dimethyl-1-[1,2,4]triazol-1-ylmethylcyclopentanol (GB 857 383); myclobutanil, 2-(4-chlorophenyl)-2-[1,2,4]triazol-1-ylmethylpentanenitrile (CAS RN 88671-89-0); penconazole, 1-[2-(2,4-dichlorophenyl)pentyl]-1H-[1,2,4]triazole (Pesticide Manual, 12th Ed. (2000), p. 712); propiconazole, 1-[[2-(2,4-dichlorophenyl)-4-propyl-1,3-dioxolan-2-yl]methyl]-1H-1,2,4-triazole (BE 835 579); prothioconazole, 2-[2-(1-chlorocyclopropyl)-3-(2-chlorophenyl)-2-hydroxypropyl]-2,4-dihydro-[1,2,4]triazole-3-thione (WO 96/16048); simeconazole, α-(4-fluorophenyl)-α-[(trimethylsilyl)methyl]-1H-1,2,4-triazole-1-ethanol [CAS RN 149508-90-7]; triadimefon, 1-(4-chlorophenoxy)-3,3-dimethyl-1-(1H-1,2,4-triazol-1-yl)-2-butanone; triadimenol, β-(4-chlorophenoxy)-α-(1,1-dimethylethyl)-1H-1,2,4-triazole-1-ethanol; tebuconazole, 1-(4-chlorophenyl)-4,4-dimethyl-3-[1,2,4]triazol-1-ylmethylpentan-3-ol (EP-A 40 345); tetraconazole, 1-[2-(2,4-dichlorophenyl)-3-(1,1,2,2-tetrafluoroethoxy)propyl]-1H-1,2,4-triazole (EP 234 242); triticonazole, (5E)-5-[(4-chlorophenyl)methylene]-2,2-dimethyl-1-(1H-1,2,4-triazol-1-ylmethyl)cyclopentanol (FR 26 41 277); prochloraz, N-{propyl-[2-(2,4,6-trichlorophenoxy)ethyl]}imidazole-1-carboxamide (U.S. Pat. No. 3,991,071); pefurazoate, 4-pentenyl 2-[(2-furanylmethyl)(1H-imidazol-1-ylcarbonyl)amino]butanoate [CAS RN 101903-30-4]; triflumizole, (4-chloro-2-trifluoromethylphenyl)-(2-propoxy-1-[1,2,4]triazol-1-ylethylidene)amine (JP-A 79/119 462); cyazofamid, 4-chloro-2-cyano-N,N-dimethyl-5-(4-methylphenyl)-1H-imidazole-1-sulfonamide (CAS RN 120116-88-3]; benomyl, N-butyl-2-acetylaminobenzoimidazol-1-carboxamide (U.S. Pat. No. 3,631,176); carbendazim, methyl (1H-benzoimidazol-2-yl)-carbamate (U.S. Pat. No. 3,657,443); thiabendazole, 2-(1,3-thiazol-4-yl)benzimidazole (U.S. Pat. No. 3,017,415); fuberidazole, 2-(2-furanyl)-1H-benzimidazole (DE 12 09 799); ethaboxam, N-(cyano-2-thienylmethyl)-4-ethyl-2-(ethylamino)-5-thiazolcarboxamide (EP-A 639 574); etridiazole; hymexazole, 5-methyl-1,2-oxazol-3-ol (JP 518249, JP 532202); azoxystrobin, methyl 2-{2-[6-(2-cyano-1-vinylpenta-1,3-dienyloxy)pyrimidin-4-yloxy]phenyl}-3-methoxyacrylate (EP-A 382 375); dimoxystrobin, (E)-2-(methoxyimino)-N-methyl-2-[α-(2,5-xylyloxy)-o-tolyl]acetamide (EP-A 477 631); fluoxastrobin, (E)-{2-[6-(2-chlorophenoxy)-5-fluoropyrimidin-4-yloxy]phenyl}(5,6-dihydro-1,4,2-dioxazin-3-yl)methanone O-methyloxime (WO 97/27189); kresoxim-methyl, methyl (E)-methoxyimino[α-(o-tolyloxy)-o-tolyl]acetate (EP-A 253 213); metominostrobin, (E)-2-(methoxyimino)-N-methyl-2-(2-phenoxyphenyl)acetamide (EP-A 398 692); orysastrobin, (2E)-2-(methoxyimino)-2-{2-[(3E,5E,6E)-5-(methoxyimino)-4,6-dimethyl-2,8-dioxa-3,7-diazanona-3,6-dien-1-yl]phenyl}-N-methylacetamide (WO 97/15552); picoxystrobin, methyl 3-methoxy-2-[2-(6-trifluoromethylpyridin-2-yloxymethyl)phenyl]acrylate (EP-A 278 595); trifloxystrobin, methyl (E)-methoxyimino-{(E)-α-[1-(α,α,α-trifluoro-m-tolyl)ethylideneaminooxy]-o-tolyl}acetate (EP-A 460 575); carboxin, 5,6-dihydro-2-methyl-N-phenyl-1,4-oxathiin-3-carboxamide (U.S. Pat. No. 3,249,499); benalaxyl, methyl N-(phenylacetyl)-N-(2,6-xylyl)-DL-alaninate (DE 29 03 612); boscalid, 2-chloro-N-(4′-chlorobiphenyl-2-yl)nicotinamide (EP-A 545 099); fenhexamid, N-(2,3-dichloro-4-hydroxyphenyl)-1-methylcyclohexanecarboxamide (Proc. Br. Crop Prot. Conf.—Pests Dis., 1998, Vol. 2, p. 327); flutolanil,α,α,α-trifluoro-3′-isopropoxy-o-toluanilide (JP 1104514); furametpyr, 5-chloro-N-(1,3-dihydro-1,1,3-trimethyl-4-isobenzofuranyl)-1,3-dimethyl-1H-pyrazole-4-carboxamide [CAS RN 123572-88-3]; mepronil, 3′-isopropoxy-o-toluanilide (U.S. Pat. No. 3,937,840); metalaxyl, methyl N-(methoxyacetyl)-N-(2,6-xylyl)-DL-alaninate (GB 15 00 581); mefenoxam, methyl N-(2,6-dimethylphenyl)-N-(methoxyacetyl)-D-alaninate; ofurace, (RS)-α-(2-chloro-N-2,6-xylylacetamido)-γ-butyrolactone [CAS RN 58810-48-3]; oxadixyl; N-(2,6-dimethylphenyl)-2-methoxy-N-(2-oxo-3-oxazolidinyl)acetamide (GB 20 58 059); oxycarboxin, 5,6-dihydro-2-methyl-1,4-oxathiin-3-carboxanilide 4,4-dioxide (U.S. Pat. No. 3,399,214); penthiopyrad, N-[2-(1,3-dimethylbutyl)-3-thienyl]-1-methyl-3-(trifluoromethyl)-1H-pyrazole-4-carboxamide (JP 10130268); thifluzamide, N-[2,6-dibromo-4-(trifluoromethoxy)phenyl]-2-methyl-4-(trifluoromethyl)-5-thiazolecarboxamide; tiadinil, 3′-chloro-4,4′-dimethyl-1,2,3-thiadiazole-5-carboxanilide [CAS RN 223580-51-6]; dimethomorph, 3-(4-chlorophenyl)-3-(3,4-dimethoxyphenyl)-1-morpholin-4-ylpropenone (EP-A 120 321); flumorph, 3-(4-fluorophenyl)-3-(3,4-dimethoxyphenyl)-1-morpholin-4-ylpropenone (EP-A 860 438); flumetover, 2-(3,4-dimethoxyphenyl)-N-ethyl-α,α,α-trifluoro-N-methyl-p-toluamide [AGROW No. 243, 22 (1995)]; fluopicolide (picobenzamid), 2,6-dichloro-N-(3-chloro-5-trifluoromethylpyridin-2-ylmethyl)benzamide (WO 99/42447); zoxamide, (RS)-3,5-dichloro-N-(3-chloro-1-ethyl-1-methyl-2-oxopropyl)-p-toluamide [CAS RN 156052-68-5]; carpropamid, 2,2-dichloro-N-[1-(4-chlorophenyl)ethyl]-1-ethyl-3-methylcyclopropane-carboxamide [CAS RN 104030-54-8]; diclocymet, 2-cyano-N-[(1R)-1-(2,4-dichlorophenyl)ethyl]-3,3-dimethyl butanamide; mandipropamid, (RS)-2-(4-chlorophenyl)-N-[3-methoxy-4-(prop-2-ynyloxy)phenethyl]-2-(prop-2-ynyloxy)acetamide [CAS RN 374726-62-2]; fluazinam, 3-chloro-N-[3-chloro-2,6-dinitro-4-(trifluoromethyl)phenyl]-5-(trifluoromethyl)-2-pyridinamine (The Pesticide Manual, publ. The British Crop Protection Council, 10th ed. (1995), p. 474); pyrifenox, 1-(2,4-dichlorophenyl)-2-(3-pyridinyl)ethanone O-methyloxime (EP-A 49 854); bupirimate, 5-butyl-2-ethylamino-6-methyl-pyrimidin-4-yldimethylsulfamate [CAS RN 41483-43-6]; cyprodinil, (4-cyclopropyl-6-methylpyrimidin-2-yl)phenylamine (EP-A 310 550); fenarimol, (4-chlorophenyl) (2-chlorophenyl)pyrimidin-5-ylmethanol (GB 12 18 623); ferimzone, (Z)-2′-methylacetophenone 4,6-dimethylpyrimidin-2-ylhydrazone [CAS RN 89269-64-7]; mepanipyrim, (4-methyl-6-prop-1-yl)pyrimidin-2-yl)phenylamine (EP-A 224 339); nuarimol, α-(2-chlorophenyl)-α-(4-fluorophenyl)-5-pyrimidinemethanol (GB 12 18 623); pyrimethanil, 4,6-dimethylpyrimidin-2-ylphenylamine (DD-A 151 404); triforine, N,N-{piperazine-1,4-diyl-bis[(trichloromethyl)methylene]}diformamide (DE 19 01 421); fenpiclonil, 4-(2,3-dichlorophenyl)-1H-pyrrole-3-carbonitrile (Proc. 1988 Br. Crop Prot. Conf.—Pests Dis., Vol. 1, p. 65); fludioxonil, 4-(2,2-difluorobenzo[1,3]dioxol-4-yl)-1H-pyrrole-3-carbonitrile (The Pesticide Manual, publ. The British Crop Protection Council, 10th ed. (1995), p. 482); aldimorph, 4-alkyl-2,5(or 2,6)-dimethylmorpholine, comprising 65-75% of 2,6-dimethylmorpholine and 25-35% of 2,5-dimethylmorpholine, comprising more than 85% of 4-dodecyl-2,5(or 2,6)-dimethylmorpholine, where “alkyl” may also include octyl, decyl, tetradecyl or hexadecyl and where the cis/trans ratio is 1:1; dodemorph, 4-cyclo-dodecyl-2,6-dimethylmorpholine (DE 1198125); fenpropimorph, (RS)-cis-4-[3-(4-tert-butylphenyl)-2-methylpropyl]-2,6-dimethylmorpholine (DE 27 52 096); tridemorph, 2,6-dimethyl-4-tridecylmorpholine (DE 11 64 152); fenpropidin, (RS)-1-[3-(4-tert-butylphenyl)-2-methylpropyl]piperidine (DE 27 52 096); iprodione, N-isopropyl-3-(3,5-dichlorophenyl)-2,4-dioxoimidazolidine-1-carboxamide (GB 13 12 536); procymidone, N-(3,5-dichlorophenyl)-1,2-dimethylcyclopropane-1,2-dicarboximide (U.S. Pat. No. 3,903,090); vinclozolin, 3-(3,5-dichlorophenyl)-5-methyl-5-vinyloxazolidine-2,4-dione (DE-OS 22 07 576); famoxadone, (RS)-3-anilino-5-methyl-5-(4-phenoxyphenyl)-1,3-oxazolidine-2,4-dione; fenamidone, (S)-1-anilino-4-methyl-2-methylthio-4-phenylimidazolin-5-one; octhilinone; probenazole, 3-allyloxy-1,2-benzothiazole 1,1-dioxide; amisulbrom, N,N-dimethyl-3-(3-bromo-6-fluoro-2-methylindole-1-sulfonyl)-[1,2,4]triazole-1-sulfonamide (WO 03/053145); anilazine, 4,6-dichloro-N-(2-chlorophenyl)-1,3,5-triazine-2-amine (U.S. Pat. No. 2,720,480); diclomezine, 6-(3,5-dichlorophenyl)-p-tolyl)pyridazin-3(2H)-one; pyroquilon; proquinazid, 6-iodo-2-propoxy-3-propylquinazolin-4(3M-one (WO 97/48684); tricyclazole, 5-methyl-1,2,4-triazolo[3,4-b]benzothiazole (GB 14 19 121); acibenzolar-S-methyl, methyl benzo[1,2,3]thiadiazole-7-carbothionate; captafol, N-(1,1,2,2-tetrachloroethylthio)cyclohex-4-ene-1,2-dicarboximide; captan, 2-trichloromethylsulfanyl-3a,4,7,7a-tetrahydroisoindole-1,3-dione (U.S. Pat. No. 2,553,770); dazomet, 3,5-dimethyl-1,3,5-thiadiazinane-2-thione; folpet, 2-trichloromethylsulfanylisoindole-1,3-dione (U.S. Pat. No. 2,553,770); fenoxanil, N-(1-cyano-1,2-dimethylpropyl)-2-(2,4-dichlorophenoxy) propanamide; quinoxyfen, 5,7-dichloro-4-(4-fluorophenoxy)quinoline (U.S. Pat. No. 5,240,940); mancozeb, manganese ethylenebis(dithiocarbanate) zinc complex (U.S. Pat. No. 3,379,610); maneb, manganese ethylenebis(dithiocarbamate) (U.S. Pat. No. 2,504,404); metam, methyldithiocarbaminic acid (U.S. Pat. No. 2,791,605); metiram, zinc ammoniate ethylenebis(dithiocarbamate) (U.S. Pat. No. 3,248,400); propineb, zinc propylenebis(dithiocarbamate) polymer (BE 611 960); ferbam, iron(3+) dimethyldithiocarbamate (U.S. Pat. No. 1,972,961); thiram, bis(dimethylthiocarbamoyl) disulfide (DE 642 532); ziram, dimethyldithiocarbamate; zineb, zinc ethylenebis(dithiocarbamate) (U.S. Pat. No. 2,457,674); diethofencarb, isopropyl 3,4-diethoxycarbanilate; iprovalicarb, isopropyl [(1S)-2-methyl-1-(1-p-tolylethylcarbamoyl)propyl]carbamate (EP-A 472 996); flubenthiavalicarb (benthiavalicarb), isopropyl {(S)-1-[(1R)-1-(6-fluorobenzothiazol-2-yl)ethylcarbamoyl]-2-methylpropyl}carbamate (JP-A 09/323,984); propamocarb, propyl 3-(dimethylamino)propylcarbamate (DE 16 43 040); dodine, (2,4-dichlorophenoxy)acetic acid (U.S. Pat. No. 2,867,562); iminoctadine, bis(8-guanidinooctyl)amine (GB 11 14 155); guazatine, mixture of products from the amidation of iminodi(octamethylene)diamine, mainly iminoctadine; kasugamycin, 1L-1,3,4/2,5,6-1-deoxy-2,3,4,5,6-pentahydroxycyclohexyl 2-amino-2,3,4,6-tetradeoxy-4-(α-iminoglycino)-α-D-arabinohexopyranoside; streptomycin, 0-2-deoxy-2-methylamino-α-L-glucopyranosyl-(1→2)-O-5-deoxy-3-C-formyl-α-L-lyxofuranosyl-(1→4)N¹,N³-diamidino-D-streptamine; polyoxins, 5-(2-amino-5-O-carbamoyl-2-deoxy-L-xylonamido)-1-(5-carboxy-1,2,3,4-tetrahydro-2,4-dioxopyrimidin-1-yl)-1,5-dideoxy-β-D-allofuranuronic acid and the salts thereof; validamycin A, binapacryl, (RS)-2-sec-butyl-4,6-dinitrophenyl 3-methylcrotonate; dinocap, the mixture of 2,6-dinitro-4-octylphenyl crotonate and 2,4-dinitro-6-octylphenyl crotonate, wherein “octyl” is a mixture of 1-methylheptyl, 1-ethylhexyl and 1-propylpentyl (U.S. Pat. No. 2,526,660); dinobuton, (RS)-2-seo-butyl-4,6-dinitrophenyl isopropyl carbonate; dithianon, 5,10-dioxo-5,10-dihydronaphtho[2,3-b][1,4]dithiin-2,3-dicarbonitrile (GB 857 383); isoprothiolane, indol-3-ylacetic acid; fentin acetate, triphenyltin acetate (U.S. Pat. No. 3,499,086); edifenphos, O-ethyl S,S-diphenyl phosphorodithioate; iprobenfos, S-benzyl O,O-diisopropyl phosphorothioate (Jpn. Pesticide Inf., No. 2, S. 11 (1970)); fosetyl, fosetyl-aluminum, (aluminum) ethylphosphonate (FR 22 54 276); pyrazophos, ethyl 2-diethoxyphosphinothioyloxy-5-methylpyrazolo[1,5-a]pyrimidine-6-carboxylate (DE 15 45 790); tolclofos-methyl, O-2,6-dichloro-p-tolyl O,O-dimethyl phosphorothioate (GB 14 67 561); chlorothalonil, 2,4,5,6-tetrachloroisophthalonitrile (U.S. Pat. No. 3,290,353); dichlofluanid, N-dichlorofluoromethylthio-N,N-dimethyl-N-phenylsulfamide (DE 11 93 498); flusulfamide, 2′,4-dichloro-α,α,α-trifluoro-4′-nitro-m-toluenesulfanilide (EP-A 199 433); hexachlorobenzene (C. R. Seances Acad. Agric. Fr., Vol. 31, p. 24 (1945)); phthalide (DE 16 43 347); pencycuron, 1-(4-chlorobenzyl)-1-cyclopentyl-3-phenylurea (DE 27 32 257); quintozene, pentachloronitrobenzene (DE 682 048); thiophanate-methyl, 1,2-phenylenebis(iminocarbonothioyl)bis(dimethylcarbamate) (DE-OS 19 30 540); tolylfluanid, N-dichlorofluoromethylthio-N,N-dimethyl-N-p-tolylsulfamide (DE 11 93 498); Bordeaux mixture, mixture of calcium hydroxide and copper(II) sulfate; copper hydroxide, Cu(OH)₂; copper oxychloride, Cu2Cl(OH)₃; cyflufenamid, (Z)-N-[α-(cyclopropylmethoxyimino)-2,3-difluoro-6-(trifluoromethyl)benzyl]-2-phenylacetamide (WO 96/19442); cymoxanil, 1-(2-cyano-2-methoxyiminoacetyl)-3-ethylurea (U.S. Pat. No. 3,957,847); dimethirimol, 5-butyl-2-dimethylamino-6-methylpyrimidin-4-ol (GB 11 82 584); ethirimol, 5-butyl-2-ethylamino-6-methylpyrimidin-4-ol (GB 11 82 584); furalaxyl, methyl N-(2-furoyl)-N-(2,6-xylyl)-DL-alaninate (GB 14 48 810); metrafenone, 3′-bromo-2,3,4,6′-tetramethoxy-2′,6-dimethylbenzophenone (U.S. Pat. No. 5,945,567); spiroxamine, (8-tert-butyl-1,4-dioxaspiro[4.5]dec-2-yl)diethylamine (EP-A 281 842).

The compounds named according to IUPAC, their preparation and their fungicidal activity are likewise known: methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate (EP-A 12 01 648); methyl 2-(ortho-((2,5-dimethylphenyloxymethylene)phenyl)-3-methoxyacrylate (EP-A 226 917); 5-chloro-7-(4-methylpiperidin-1-yl)-6-(2,4,6-trifluorophenyl)-[1,2,4]triazolo[1,5-a]pyrimidine (WO 98/46608), 3,4-dichloro-N-(2-cyanophenyl)isothiazole-5-carboxamide (WO 99/24413), N-(2-{4-[3-(4-chlorophenyl)prop-2-ynyloxy]-3-methoxyphenyl}ethyl)-2-methanesulfonylamino-3-methylbutyramide, N-(2-{4-[3-(4-chlorophenyl)prop-2-ynyloxy]-3-methoxyphenyl}ethyl)-2-ethane-sulfonylamino-3-methylbutyramide (WO 04/049804), N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide (WO 03/066609); N-(3′,4′-dichloro-5-fluorobiphenyl-2-yl)-3-difluoromethyl-1-methylpyrazole-4-carboxamide (WO 03/053145); 2-butoxy-6-iodo-3-propylchromen-4-one (WO 03/14103); 3-[5-(4-chlorophenyl)-2,3-dimethylisoxazolidin-3-yl]pyridine (EP-A 10 35 122); amisulbrom, N,N-dimethyl-3-(3-bromo-6-fluoro-2-methylindole-1-sulfonyl)-[1,2,4]triazole-1-sulfonamide (WO 03/053145); methyl 3-(4-chlorophenyl)-3-(2-isopropoxycarbonylamino-3-methyl-butyrylamino)propanoate (EP-A 1028125).

6-(3,4-dichloro-phenyl)-5-methyl-[1,2,4]triazolo[1,5-a]pyrimidine-7-ylamine, 6-(4-tert-butylphenyl)-5-methyl-[1,2,4]triazolo[1,5-a]pyrimidine-7-ylamine, 5-methyl-6-(3,5,5-trimethyl-hexyl)-[1,2,4]triazolo[1,5-a]pyrimidine-7-ylamine, 5-methyl-6-octyl-[1,2,4]triazolo[1,5-a]pyrimidine-7-ylamine, 6-methyl-5-octyl-[1,2,4]triazolo[1,5-a]pyrimidine-7-ylamine, 6-ethyl-5-octyl-[1,2,4]triazolo[1,5-a]pyrimidine-7-ylamine, 5-ethyl-6-octyl-[1,2,4]triazolo[1,5-a]pyrimidine-7-ylamine, 5-ethyl-6-(3,5,5-trimethyl-hexyl)-[1,2,4]triazolo[1,5-a]pyrimidine-7-ylamine, 6-octyl-5-propyl-[1,2,4]triazolo[1,5-a]pyrimidine-7-ylamine, 5-methoxymethyl-6-octyl-[1,2,4]triazolo[1,5-a]pyrimidine-7-yl-amine, 6-octyl-5-trifluoromethyl-[1,2,4]triazolo[1,5-a]pyrimidine-7-ylamine and 5-trifluoromethyl-6-(3,5,5-trimethyl-hexyl)-[1,2,4]triazolo[1,5-a]pyrimidine-7-ylamine are known from EP-A 71 792; EP-A 141 317; WO 03/009687; WO 05/087771; WO 05/087772 (=PCT/EP/05/002426); WO 05/087773; PCT/EP2006/050922 (=WO2006/087325); and/or PCT/EP2006/060399 (=WO 2006/092428).

N-(2′,4′-difluorobiphenyl-2-yl)-1-methyl-3-trifluoromethyl-1H-pyrazole-4-carboxamide; N-(2′,4′-dichlorobiphenyl-2-yl)-1-methyl-3-trifluoromethyl-1H-pyrazole-4-carboxamide; N-(2′,4′-difluorobiphenyl-2-yl)-3-difluoromethyl-1-methyl-1H-pyrazole-4-carboxamide; N-(2′,4′-dichlorobiphenyl-2-yl)-3-difluoromethyl-1-methyl-1H-pyrazole-4-carboxamide; N-(2′,5′-difluorobiphenyl-2-yl)-1-methyl-3-trifluoromethyl-1H-pyrazole-4-carboxamide; N-(2′,5′-dichlorobiphenyl-2-yl)-1-methyl-3-trifluoromethyl-1H-pyrazole-4-carboxamide; N-(2′,5′-difluorobiphenyl-2-yl)-3-difluoromethyl-1-methyl-1H-pyrazole-4-carboxamide; N-(2′,5′-dichlorobiphenyl-2-yl)-3-difluoromethyl-1-methyl-1H-pyrazole-4-carboxamide; N-(3′,5′-difluorobiphenyl-2-yl)-1-methyl-3-trifluoromethyl-1H-pyrazole-4-carboxamide; N-(3′,5′-dichlorobiphenyl-2-yl)-1-methyl-3-trifluoromethyl-1H-pyrazole-4-carboxamide, N-(3′,5′-difluorobiphenyl-2-yl)-3-difluoromethyl-1-methyl-1H-pyrazole-4-carboxamide; N-(3′,5′-dichlorobiphenyl-2-yl)-3-difluoromethyl-1-methyl-1H-pyrazole-4-carboxamide; N-(3′-fluorobiphenyl-2-yl)-1-methyl-3-trifluoromethyl-1H-pyrazole-4-carboxamide; N-(3′-chlorobiphenyl-2-yl)-1-methyl-3-trifluoromethyl-1H-pyrazole-4-carboxamide; N-(3′-fluorobiphenyl-2-yl)-3-difluoromethyl-1-methyl-1H-pyrazole-4-carboxamide; N-(3′-chlorobiphenyl-2-yl)-3-difluoromethyl-1-methyl-1H-pyrazole-4-carboxamide; N-(2′-fluorobiphenyl-2-yl)-1-methyl-3-trifluoromethyl-1H-pyrazole-4-carboxamide; N-(2′-chlorobiphenyl-2-yl)-1-methyl-3-trifluoromethyl-1H-pyrazole-4-carboxamide; N-(2′-fluorobiphenyl-2-yl)-3-difluoromethyl-1-methyl-1H-pyrazole-4-carboxamide; N-(2′-chlorbiphenyl-2-yl)-3-difluoromethyl-1-methyl-1H-pyrazole-4-carboxamide; N-(2′-fluoro-4′-chloro-5′-methylbiphenyl-2-yl)-1-methyl-3-trifluoromethyl-1H-pyrazole-4-carboxamide; N-(3′,4′,5′-trifluorobiphenyl-2-yl)-1-methyl-3-trifluoromethyl-1H-pyrazole-4-carboxamide; N-(3′,4′,5′-trifluorobiphenyl-2-yl)-1-methyl-3-difluoromethyl-1H-pyrazole-4-carboxamide; N-(2′,4′,5′-trifluorobiphenyl-2-yl)-1-methyl-3-difluoromethyl-1H-pyrazole-4-carboxamide; N-(3′,4′,5′-trifluorobiphenyl-2-yl)-3-chlorofluoromethyl-1-methyl-1H-pyrazole-4-carboxamide; N-[2-(1,1,2,3,3,3-hexafluoropropoxy)phenyl]-1-methyl-3-trifluoromethyl-1H-pyrazole-4-carboxamide; N-[2-(1,1,2,3,3,3-hexafluoropropoxy)-phenyl]-3-difluoromethyl-1-methyl-1H-pyrazole-4-carboxamide, N-[2-(2-chloro-1,1,2-trifluoroethoxy)phenyl]-1-methyl-3-trifluoromethyl-1H-pyrazole-4-carboxamide; N-[2-(2-chlor-1,1,2-trifluoroethoxy)phenyl]3-difluoromethyl-1-methyl-1H-pyrazole-4-carboxamide; N-[2-(1,1,2,2-tetrafluoroethoxy)phenyl]-3-difluoromethyl-1-methyl-1H-pyrazole-4-carboxamide; N-[2-(1,1,2,2-tetrafluoroethoxy)phenyl]-1-methyl-3-trifluoromethyl-1H-pyrazole-4-carboxamide; N-(4′-(trifluoromethylthio)biphenyl-2-yl)-3-difluoromethyl-1-methyl-1H-pyrazole-4-carboxamide; and N-(4′-(trifluoromethylthio)biphenyl-2-yl)-1-methyl-3-trifluoromethyl-1H-pyrazole-4-carboxamide are known from WO 2006/087343, WO 2001/42223, WO 2005/34628, WO 2005/123689, WO 2005/123690, WO 2006/120219, PCT/EP2006/064991 (=WO 2007/017450), and/or WO 2008/053044 (the earlier EP application no. 06123463.9).

In the mixtures of the present invention, the at least one compound I and the at least one compound II are present in synergistically effective amounts. This means that the relative amount, i.e. the weight ratio of the at least one compound I and the at least one compound II in the mixture provides for an increased fungicidal efficacy which exceeds the additive fungicidal efficacy of the compounds of the mixture as calculated from the fungicidal efficacy of the individual compounds at a given application rate. The calculation of the additive fungicidal efficacies can be performed e.g. by Colby's formula (Colby, S. R. “Calculating synergistic and antagonistic responses of herbicide Combinations”, Weeds, 15, 20-22, 1967). Synergism is present if the observed efficacy is greater than the calculated efficacy.

To ensure synergism, the at least one compound of the formula I and the at least one compound II are usually present In the mixtures of the present invention in a weight ratio of from 500:1 to 1:100, preferably from 100:1 to 1:100, more preferably from 50:1 to 1:50, in particular from 20:1 to 1:20.

According to the present invention, preference is given to secondary and tertiary mixtures. Secondary mixtures are those mixtures which contain at least one active compound of the formula I, in particular exactly one compound of the formula I and one fungicidal compound of the group II of fungicidal compounds.

Ternary mixtures are those mixtures which contain at least one active compound of the formula I, in particular exactly one compound of the formula I and two different fungicidal compounds of the group II of fungicidal compounds, hereinafter also referred to as compounds IIa and IIb.

In the binary mixtures of the present invention the at least one compound of the formula I and the one compound II are usually present in a weight ratio of from 500:1 to 1:100, preferably from 100:1 to 1:100, more preferably from 50:1 to 1:50, in particular from 20:1 to 1:20.

In the ternary mixtures of the present invention the at least one compound of the formula I and the two compounds IIa and IIb are usually present in a weight ratio of compound Ito compounds IIa+IIb of from 500:1 to 1:100, preferably from 100:1 to 1:100, more preferably from 50:1 to 1:50, in particular from 20:1 to 1:20. The weight ratio of the first compound IIa to the second compound IIb is usually in the range from 100:1 to 1:100, more preferably from 50:1 to 1:50, in particular from 20:1 to 1:20.

Preferred are secondary mixtures containing a compound I and a fungicidal compound II selected from the list comprising azoles: cyproconazole, difenoconazole, epoxiconazole, fenbuconazole, fluquinconazole, flutriafol, hexaconazole, ipconazole, metconazole, propiconazole, prothioconazole, tebuconazole, tetraconazole, triadimenol, triadimefon, triticonazole, cyazofamid, imazalil, prochloraz, triflumizol, benomyl, carbendazim, thiabendazole, ethaboxam, and hymexazole. Preference is likewise given to secondary mixtures containing a compound I and carbendazim as fungicidal compound II selected from the list comprising azoles. Especially preferred are secondary mixtures containing a compound I and a fungicidal compound II selected from the list comprising azoles: carbendazim, prothioconazole and tebuconazole.

Especially preferred are secondary mixtures containing a compound I and a fungicidal compound II selected from the list comprising strobilurins: azoxystrobin, dimoxystrobin, fluoxastrobin, kresoxim-methyl, metominostrobin, picoxystrobin, and trifloxystrobin; in particular dimoxystrobin.

Especially preferred are secondary mixtures containing a compound I and a fungicidal compound II selected from the list comprising carboxamides: boscalid, carboxin, benalaxyl, fenhexamid, flutolanil, furametpyr, metalaxyl, mefenoxam (metalaxyl-M), ofurace, oxadixyl, oxycarboxin, penthiopyrad, thifluzamide, tiadinil, dimethomorph, fluopicolide (picobenzamid), and diclocymet.

Especially preferred are secondary mixtures containing a compound I and a fungicidal compound II selected from the list comprising heterocylic compounds: pyrimethanil, fenpiclonil, fludioxonil, aldimorph, dodemorph, fenpropidin, cyprodinil, fenpropimorph, tridemorph, iprodione, procymidone, famoxadone, fenamidone, octhilinone, probenazole, diclomezine, pyroquilon, proquinazid, tricyclazole, captafol, captan, dazomet, fenoxanil, and quinoxyfen. Especially preferred are also secondary mixtures containing a compound I and a fungicidal compound II selected from the list comprising heterocylic compounds: 5-ethyl-6-octyl-[1,2,4]triazolo[1,5-a]pyrimidine-7-ylamine. In particular preferred are secondary mixtures containing a compound I and a fungicidal compound II selected from the list comprising heterocylic compounds: captan, 5-ethyl-6-octyl-[1,2,4]triazolo[1,5-a]pyrimidine-7-ylamine, fenpropimorph and tridemorph.

Especially preferred are secondary mixtures containing a compound I and a fungicidal compound II selected from the list comprising carbamates: mancozeb, maneb, metam, metiram, ferbam, propineb, thiram, zineb, ziram, thiophanate-methyl;

diethofencarb, iprovalicarb, propamocarb, and methyl 3-(4-chlorophenyl)-3-(2-isopropoxycarbonylamino-3-methylbutyrylamino)propanoate. In particular preferred are secondary mixtures containing a compound I and a fungicidal compound II selected from the list comprising carbamates: iprovalicarb and propineb.

Especially preferred are secondary mixtures containing a compound I and a fungicidal compound II selected from the list comprising: guazatine; streptomycin, validamycin A; binapacryl, dinocap, dinobuton; dithianon, isoprothiolane; fentin salts, such as fentin acetate; edifenphos, iprobenfos, fosetyl, pyrazophos, chlorothalonil, dichlofluanid, flusulfamide, phthalide, quintozene, tolylfluanid; copper acetate, copper hydroxide, copper oxychloride, basic copper sulfate, sulfur; cyflufenamid, cymoxanil, dimethirimol, ethirimol, furalaxyl, metrafenone and spiroxamine. In particular preferred are secondary mixtures containing a compound I and a fungicidal compound II selected from the list comprising chlorothalonil.

TABLE B

| Mixture | Component 1 | Component 2 |
|---|---|---|
| B-1 | one compound of formula I | Azoxystrobin |
| B-2 | one compound of formula I | Dimoxystrobin |
| B-3 | one compound of formula I | Enestroburin |
| B-4 | one compound of formula I | Fluoxastrobin |
| B-5 | one compound of formula I | Kresoxim-methyl |
| B-6 | one compound of formula I | Metominostrobin |
| B-7 | one compound of formula I | Orysastrobin |
| B-8 | one compound of formula I | Picoxystrobin |
| B-9 | one compound of formula I | Pyraclostrobin |
| B-10 | one compound of formula I | Pyribencarb |
| B-11 | one compound of formula I | Trifloxystrobin |
| B-12 | one compound of formula I | 2-(2-(6-(3-Chloro-2-methyl-phenoxy)-5-fluoro- |
| | | pyrimidine-4-yloxy)-phenyl)-2- |
| | | methoxyimino-N-methyl-acetamide |
| B-13 | one compound of formula I | 2-(ortho-((2,5-Dimethylphenyl-oxymethylen)phenyl)- |
| | | 3-methoxy-acrylic acidmethyl- |
| | | ester |
| B-14 | one compound of formula I | 3-Methoxy-2-(2-(N-(4-methoxy-phenyl)- |
| | | cyclopropancarboximidoylsulfanylmethyl)- |
| | | phenyl)-acrylic acidmethylester |
| B-15 | one compound of formula I | Benalaxyl |
| B-16 | one compound of formula I | Benalaxyl-M |
| B-17 | one compound of formula I | Benodanil |
| B-18 | one compound of formula I | Bixafen |
| B-19 | one compound of formula I | Boscalid |
| B-20 | one compound of formula I | Carboxin |
| B-21 | one compound of formula I | Fenfuram |
| B-22 | one compound of formula I | Fenhexamid |
| B-23 | one compound of formula I | Flutolanil |
| B-24 | one compound of formula I | Furametpyr |
| B-25 | one compound of formula I | Isotianil |
| B-26 | one compound of formula I | Kiralaxyl |
| B-27 | one compound of formula I | Mepronil |
| B-28 | one compound of formula I | Metalaxyl |
| B-29 | one compound of formula I | Ofurace |
| B-30 | one compound of formula I | Oxadixyl |
| B-31 | one compound of formula I | Oxycarboxin |
| B-32 | one compound of formula I | Penthiopyrad |
| B-33 | one compound of formula I | Thifluzamide |
| B-34 | one compound of formula I | Tecloftalam |
| B-35 | one compound of formula I | Tiadinil |
| B-36 | one compound of formula I | 2-Amino-4-methyl-thiazole-5-carboxylic |
| | | acid anilide |
| B-37 | one compound of formula I | 2-Chloro-N-(1,1,3-trimethyl-indan-4-yl)- |
| | | nicotinamide |
| B-38 | one compound of formula I | N-(3′,4′-Dichloro-5-fluoro-biphenyl-2-yl)-3- |
| | | difluoromethyl-1-methyl-1H-pyrazole-4- |
| | | carboxylic acid amide |
| B-39 | one compound of formula I | N-[2-(1,3-dimethyl-butyl)-phenyl]-1,3- |
| | | dimethyl-5-fluoro-1H-pyrazole-4-carboxylic |
| | | acid amide |
| B-40 | one compound of formula I | N-(4′-Chloro-3′,5-difluoro-biphenyl-2-yl)-3- |
| | | difluoromethyl-1-methyl-1H-pyrazole-4- |
| | | carboxylic acid amide |
| B-41 | one compound of formula I | N-(4′-Chloro-3′,5-difluoro-biphenyl-2-yl)-3- |
| | | trifluoromethyl-1-methyl-1H-pyrazole-4- |
| | | carboxylic acid amide |
| B-42 | one compound of formula I | N-(3′,4′-Dichloro-5-fluoro-biphenyl-2-yl)-3- |
| | | trifluoromethyl-1-methyl-1H-pyrazole-4- |
| | | carboxylic acid amide |
| B-43 | one compound of formula I | N-(3′,5-Difluoro-4′-methyl-biphenyl-2-yl)-3- |
| | | difluoromethyl-1-methyl-1H-pyrazole-4- |
| | | carboxylic acid amide |
| B-44 | one compound of formula I | N-(3′,5-Difluoro-4′-methyl-biphenyl-2-yl)-3- |
| | | trifluoromethyl-1-methyl-1H-pyrazole-4- |
| | | carboxylic acid amide |
| B-45 | one compound of formula I | N-(2-Bicyclopropyl-2-yl-phenyl)-3-difluoro- |
| | | methyl-1-methyl-1H-pyrazole-4-carboxylic |
| | | acid amide |
| B-46 | one compound of formula I | N-(cis-2-Bicyclopropyl-2-yl-phenyl)-3-di- |
| | | fluoromethyl-1-methyl-1H-pyrazole-4-carboxylic |
| | | acid amide |
| B-47 | one compound of formula I | N-(trans-2-Bicyclopropyl-2-yl-phenyl)-3- |
| | | difluoromethyl-1-methyl-1H-pyrazole-4- |
| | | carboxylic acid amide |
| B-48 | one compound of formula I | Dimethomorph |
| B-49 | one compound of formula I | Flumorph |
| B-50 | one compound of formula I | Flumetover |
| B-51 | one compound of formula I | Fluopicolide (Picobenzamid) |
| B-52 | one compound of formula I | Fluopyram |
| B-53 | one compound of formula I | Zoxamide |
| B-54 | one compound of formula I | N-(3-Ethyl-3,5,5-trimethyl-cyclohexyl)-3- |
| | | formylamino-2-hydroxy-benzamide |
| B-55 | one compound of formula I | Carpropamid |
| B-56 | one compound of formula I | Diclocymet |
| B-57 | one compound of formula I | Mandipropamid |
| B-58 | one compound of formula I | Oxytetracyclin |
| B-59 | one compound of formula I | Silthiofam |
| B-60 | one compound of formula I | N-(6-methoxy-pyridin-3-yl)cyclopropane- |
| | | carboxylic acid amide |
| B-61 | one compound of formula I | Azaconazole |
| B-62 | one compound of formula I | Bitertanol |
| B-63 | one compound of formula I | Bromuconazole |
| B-64 | one compound of formula I | Cyproconazole |
| B-65 | one compound of formula I | Difenoconazole |
| B-66 | one compound of formula I | Diniconazole |
| B-67 | one compound of formula I | Diniconazole-M |
| B-68 | one compound of formula I | Enilconazole |
| B-69 | one compound of formula I | Epoxiconazole |
| B-70 | one compound of formula I | Fenbuconazole |
| B-71 | one compound of formula I | Flusilazole |
| B-72 | one compound of formula I | Fluquinconazole |
| B-73 | one compound of formula I | Flutriafol |
| B-74 | one compound of formula I | Hexaconazol |
| B-75 | one compound of formula I | Imibenconazole |
| B-76 | one compound of formula I | Ipconazole |
| B-77 | one compound of formula I | Metconazol |
| B-78 | one compound of formula I | Myclobutanil |
| B-79 | one compound of formula I | Oxpoconazol |
| B-80 | one compound of formula I | Paclobutrazol |
| B-81 | one compound of formula I | Penconazole |
| B-82 | one compound of formula I | Propiconazole |
| B-83 | one compound of formula I | Prothioconazole |
| B-84 | one compound of formula I | Simeconazole |
| B-85 | one compound of formula I | Tebuconazole |
| B-86 | one compound of formula I | Tetraconazole |
| B-87 | one compound of formula I | Triadimenol |
| B-88 | one compound of formula I | Triadimefon |
| B-89 | one compound of formula I | Triticonazole |
| B-90 | one compound of formula I | Uniconazole |
| B-91 | one compound of formula I | 1-(4-Chloro-phenyl)-2-([1,2,4]triazol-1-yl)- |
| | | cycloheptanol |
| B-92 | one compound of formula I | Cyazofamid |
| B-93 | one compound of formula I | Imazalil |
| B-94 | one compound of formula I | Imazalil-sulfat |
| B-95 | one compound of formula I | Pefurazoate |
| B-96 | one compound of formula I | Prochloraz |
| B-97 | one compound of formula I | Triflumizole |
| B-98 | one compound of formula I | Benomyl |
| B-99 | one compound of formula I | Carbendazim |
| B-100 | one compound of formula I | Fuberidazole |
| B-101 | one compound of formula I | Thiabendazole |
| B-102 | one compound of formula I | Ethaboxam |
| B-103 | one compound of formula I | Etridiazole |
| B-104 | one compound of formula I | Hymexazole |
| B-105 | one compound of formula I | Fluazinam |
| B-106 | one compound of formula I | Pyrifenox |
| B-107 | one compound of formula I | 1-(4-Chloro-phenyl)-1-(propyn-2-yloxy)-3- |
| | | (4-(3,4-dimethoxy-phenyl)-isoxazol-5-yl)- |
| | | propane-2-one |
| B-108 | one compound of formula I | 3-[5-(4-Chloro-phenyl)-2,3-dimethyl-isoxazolidine- |
| | | 3-yl]-pyridine |
| B-109 | one compound of formula I | 2,3,5,6-Tetrachloro-4- |
| | | methanesulfonylpyridine |
| B-110 | one compound of formula I | 3,4,5-Trichloro-pyridine-2,6-dicarbonitrile |
| B-111 | one compound of formula I | N-(1-(5-Bromo-3-chloro-pyridine-2-yl)- |
| | | ethyl)-2,4-dichloro-nicotinamide |
| B-112 | one compound of formula I | N-((5-Bromo-3-chloro-pyridine-2-yl)- |
| | | methyl)-2,4-dichloro-nicotinamide |
| B-113 | one compound of formula I | Bupirimate |
| B-114 | one compound of formula I | Cyprodinil |
| B-115 | one compound of formula I | Diflumetorim |
| B-116 | one compound of formula I | Ferimzone |
| B-117 | one compound of formula I | Fenarimol |
| B-118 | one compound of formula I | Mepanipyrim |
| B-119 | one compound of formula I | Nitrapyrin |
| B-120 | one compound of formula I | Nuarimol |
| B-121 | one compound of formula I | Pyrimethanil |
| B-122 | one compound of formula I | Fludioxonil |
| B-123 | one compound of formula I | Fenpiclonil |
| B-124 | one compound of formula I | Aldimorph |
| B-125 | one compound of formula I | Dodemorph |
| B-126 | one compound of formula I | Dodemorph acetate |
| B-127 | one compound of formula I | Fenpropimorph |
| B-128 | one compound of formula I | Tridemorph |
| B-129 | one compound of formula I | Fluoroimid |
| B-130 | one compound of formula I | Iprodione |
| B-131 | one compound of formula I | Procymidone |
| B-132 | one compound of formula I | Vinclozolin |
| B-133 | one compound of formula I | Acibenzolar-S-methyl |
| B-134 | one compound of formula I | Amisulbrom |
| B-135 | one compound of formula I | Anilazin |
| B-136 | one compound of formula I | Blasticidin-S |
| B-137 | one compound of formula I | Captan |
| B-138 | one compound of formula I | Captafol |
| B-139 | one compound of formula I | Chinomethionat |
| B-140 | one compound of formula I | Dazomet |
| B-141 | one compound of formula I | Debacarb |
| B-142 | one compound of formula I | Diclomezine |
| B-143 | one compound of formula I | Difenzoquat |
| B-144 | one compound of formula I | Difenzoquat-methylsulphat |
| B-145 | one compound of formula I | Famoxadone |
| B-146 | one compound of formula I | Fenamidone |
| B-147 | one compound of formula I | Fenoxanil |
| B-148 | one compound of formula I | Fenpropidin |
| B-149 | one compound of formula I | Folpet |
| B-150 | one compound of formula I | Octhilinone |
| B-151 | one compound of formula I | Oxolinic acid |
| B-152 | one compound of formula I | Piperalin |
| B-153 | one compound of formula I | Probenazole |
| B-154 | one compound of formula I | Proquinazid |
| B-155 | one compound of formula I | Pyroquilon |
| B-156 | one compound of formula I | Quinoxyfen |
| B-157 | one compound of formula I | Triazoxid |
| B-158 | one compound of formula I | Tricyclazole |
| B-159 | one compound of formula I | Triforine |
| B-160 | one compound of formula I | 5-Chloro-7-(4-methyl-piperidine-1-yl)-6- |
| | | (2,4,6-trifluoro-phenyl)-[1,2,4]triazolo[1,5- |
| | | a]pyrimidine |
| B-161 | one compound of formula I | 2-Butoxy-6-iodo-3-propyl-chromen-4-one |
| B-162 | one compound of formula I | Ferbam |
| B-163 | one compound of formula I | Mancozeb |
| B-164 | one compound of formula I | Maneb |
| B-165 | one compound of formula I | Metiram |
| B-166 | one compound of formula I | Metam |
| B-167 | one compound of formula I | Methasulphocarb |
| B-168 | one compound of formula I | Propineb |
| B-169 | one compound of formula I | Thiram |
| B-170 | one compound of formula I | Zineb |
| B-171 | one compound of formula I | Ziram |
| B-172 | one compound of formula I | Diethofencarb |
| B-173 | one compound of formula I | Flubenthiavalicarb |
| B-174 | one compound of formula I | Iprovalicarb |
| B-175 | one compound of formula I | Propamocarb |
| B-176 | one compound of formula I | Propamocarb hydrochloride |
| B-177 | one compound of formula I | methyl 3-(4-chlorophenyl)-3-(2- |
| | | isopropoxycarbonylamino-3-methyl- |
| | | butyrylamino)propanoate |
| B-178 | one compound of formula I | Valiphenal |
| B-179 | one compound of formula I | 4-Fluorophenyl N-(1-(1-(4- |
| | | cyanophenyl)ethanesulfonyl)-but-2- |
| | | yl)carbamate |
| B-180 | one compound of formula I | Dodine |
| B-181 | one compound of formula I | Dodine (free base) |
| B-182 | one compound of formula I | Iminoctadine |
| B-183 | one compound of formula I | Iminoctadine triacetate |
| B-184 | one compound of formula I | Iminoctadine-tris(albesilat) |
| B-185 | one compound of formula I | Guazatine |
| B-186 | one compound of formula I | Guazatine acetate |
| B-187 | one compound of formula I | Kasugamycin |
| B-188 | one compound of formula I | Kasugamycin-hydrochlorid-hydrate |
| B-189 | one compound of formula I | Polyoxine |
| B-190 | one compound of formula I | Streptomycin |
| B-191 | one compound of formula I | Validamycin A |
| B-192 | one compound of formula I | Binapacryl |
| B-193 | one compound of formula I | Dicloran |
| B-194 | one compound of formula I | Dinobuton |
| B-195 | one compound of formula I | Dinocap |
| B-196 | one compound of formula I | Nitrothal-isopropyl |
| B-197 | one compound of formula I | Tecnazen |
| B-198 | one compound of formula I | Fentin-acetate |
| B-199 | one compound of formula I | Fentin-chloride |
| B-200 | one compound of formula I | Fentin hydroxide |
| B-201 | one compound of formula I | Isoprothiolane |
| B-202 | one compound of formula I | Dithianon |
| B-203 | one compound of formula I | Edifenphos |
| B-204 | one compound of formula I | Fosetyl |
| B-205 | one compound of formula I | Fosetyl-Aluminium |
| B-206 | one compound of formula I | Iprobenfos |
| B-207 | one compound of formula I | Pyrazophos |
| B-208 | one compound of formula I | Tolclofos-methyl |
| B-209 | one compound of formula I | Chlorothalonil |
| B-210 | one compound of formula I | Dichlofluanid |
| B-211 | one compound of formula I | Dichlorophen |
| B-212 | one compound of formula I | Flusulfamide |
| B-213 | one compound of formula I | Hexachlorbenzene |
| B-214 | one compound of formula I | Pencycuron |
| B-215 | one compound of formula I | Pentachlorophenol and its salts |
| B-216 | one compound of formula I | Phthalide |
| B-217 | one compound of formula I | Quintozene |
| B-218 | one compound of formula I | Thiophanate Methyl |
| B-219 | one compound of formula I | Tolylfluanid |
| B-220 | one compound of formula I | N-(4-Chloro-2-nitro-phenyl)-N-ethyl-4- |
| | | methyl-benzenesulfonamide |
| B-221 | one compound of formula I | phosphorous acid and its salts |
| B-222 | one compound of formula I | sulfur |
| B-223 | one compound of formula I | Bordeaux mixture |
| B-224 | one compound of formula I | copper acetate |
| B-225 | one compound of formula I | copper hydroxide |
| B-226 | one compound of formula I | copper oxychloride |
| B-227 | one compound of formula I | basic copper sulfate |
| B-228 | one compound of formula I | Biphenyl |
| B-229 | one compound of formula I | Bronopol |
| B-230 | one compound of formula I | Cyflufenamid |
| B-231 | one compound of formula I | Cymoxanil |
| B-232 | one compound of formula I | Diphenylamin |
| B-233 | one compound of formula I | Metrafenon |
| B-234 | one compound of formula I | Mildiomycin |
| B-235 | one compound of formula I | Oxin-copper |
| B-236 | one compound of formula I | Prohexadione-Calcium |
| B-237 | one compound of formula I | Spiroxamin |
| B-238 | one compound of formula I | Tolylfluanid |
| B-239 | one compound of formula I | N-(Cyclopropylmethoxyimino-(6-difluoro- |
| | | methoxy-2,3-difluoro-phenyl)-methyl)-2- |
| | | phenyl acetamide |
| B-240 | one compound of formula I | N′-(4-(4-Chloro-3-trifluoromethyl-phenoxy)- |
| | | 2,5-dimethyl-phenyl)-N-ethyl-N-methyl- |
| | | formamidine |
| B-241 | one compound of formula I | N′-(4-(4-Fluoro-3-trifluoromethyl-phenoxy)- |
| | | 2,5-dimethyl-phenyl)-N-ethyl-N-methyl- |
| | | formamidine |
| B-242 | one compound of formula I | N′-(2-Methyl-5-trifluoromethyl-4-(3-tri- |
| | | methylsilanyl-propoxy)-phenyl)-N-ethyl-N- |
| | | methylformamidine |
| B-243 | one compound of formula I | N′-(5-Difluoromethyl-2-methyl-4-(3-tri- |
| | | methylsilanyl-propoxy)-phenyl)-N-ethyl-N- |
| | | methylformamidine |
| B-244 | one compound of formula I | 5-ethyl-6-octyl-[1,2,4]triazolo[1,5-a]pyrimidine- |
| | | 7-ylamine |

Particular embodiments of the invention are mixtures B-1.1 to B-244.1, wherein the compound of the formula I is the compound 1.1 of table A and wherein the compound II is the compound given in one line of table B.

Particular embodiments of the invention are mixtures B-1.2 to B-244.2, wherein the compound of the formula I is the compound 1.2 of table A and wherein the compound II is the compound given in one line of table B.

Particular embodiments of the invention are mixtures B-1.3 to B-244.3, wherein the compound of the formula I is the compound 1.3 of table A and wherein the compound II is the compound given in one line of table B.

Particular embodiments of the invention are mixtures B-1.4 to B-244.4, wherein the compound of the formula I is the compound 1.4 of table A and wherein the compound II is the compound given in one line of table B.

Particular embodiments of the invention are mixtures B-1.5 to B-244.5, wherein the compound of the formula I is the compound 1.5 of table A and wherein the compound II is the compound given in one line of table B.

Particular embodiments of the invention are mixtures B-1.6 to B-244.6, wherein the compound of the formula I is the compound 1.6 of table A and wherein the compound II is the compound given in one line of table B.

Particular embodiments of the invention are mixtures B-1.7 to B-244.7, wherein the compound of the formula I is the compound 1.7 of table A and wherein the compound II is the compound given in one line of table B.

Particular embodiments of the invention are mixtures B-1.8 to B-244.8, wherein the compound of the formula I is the compound 1.8 of table A and wherein the compound II is the compound given in one line of table B.

Particular embodiments of the invention are mixtures B-1.9 to B-244.9, wherein the compound of the formula I is the compound 1.9 of table A and wherein the compound II is the compound given in one line of table B.

Particular embodiments of the invention are mixtures B-1.10 to B-244.10, wherein the compound of the formula I is the compound 1.10 of table A and wherein the compound II is the compound given in one line of table B.

Particular embodiments of the invention are mixtures B-1.11 to B-244.11, wherein the compound of the formula I is the compound 1.11 of table A and wherein the compound II is the compound given in one line of table B.

Particular embodiments of the invention are mixtures B-1.12 to B-244.12, wherein the compound of the formula I is the compound 1.12 of table A and wherein the compound II is the compound given in one line of table B.

Particular embodiments of the invention are mixtures B-1.13 to B-244.13, wherein the compound of the formula I is the compound 1.13 of table A and wherein the compound II is the compound given in one line of table B.

Particular embodiments of the invention are mixtures B-1.14 to B-244.14, wherein the compound of the formula I is the compound 1.14 of table A and wherein the compound II is the compound given in one line of table B.

Particular embodiments of the invention are mixtures B-1.15 to B-244.15, wherein the compound of the formula I is the compound 1.15 of table A and wherein the compound II is the compound given in one line of table B.

Particular embodiments of the invention are mixtures B-1.16 to B-244.16, wherein the compound of the formula I is the compound 1.16 of table A and wherein the compound II is the compound given in one line of table B.

Particular embodiments of the invention are mixtures B-1.17 to B-244.17, wherein the compound of the formula I is the compound 1.17 of table A and wherein the compound II is the compound given in one line of table B.

Particular embodiments of the invention are mixtures B-1.18 to B-244.18, wherein the compound of the formula I is the compound 1.18 of table A and wherein the compound II is the compound given in one line of table B.

Particular embodiments of the invention are mixtures B-1.19 to B-244.19, wherein the compound of the formula I is the compound 1.19 of table A and wherein the compound II is the compound given in one line of table B.

Particular embodiments of the invention are mixtures B-1.20 to B-244.20, wherein the compound of the formula I is the compound 1.20 of table A and wherein the compound II is the compound given in one line of table B.

Particular embodiments of the invention are mixtures B-1.21 to B-244.21, wherein the compound of the formula I is the compound 1.21 of table A and wherein the compound II is the compound given in one line of table B.

Particular embodiments of the invention are mixtures B-1.22 to B-244.22, wherein the compound of the formula I is the compound 1.22 of table A and wherein the compound II is the compound given in one line of table B.

Particular embodiments of the invention are mixtures B-1.23 to B-244.23, wherein the compound of the formula I is the compound 1.23 of table A and wherein the compound II is the compound given in one line of table B.

Particular embodiments of the invention are mixtures B-1.24 to B-244.24, wherein the compound of the formula I is the compound 1.24 of table A and wherein the compound II is the compound given in one line of table B.

Particular embodiments of the invention are mixtures B-1.25 to B-244.25, wherein the compound of the formula I is the compound 1.25 of table A and wherein the compound II is the compound given in one line of table B.

Particular embodiments of the invention are mixtures B-1.26 to B-244.26, wherein the compound of the formula I is the compound 1.26 of table A and wherein the compound II is the compound given in one line of table B.

Particular embodiments of the invention are mixtures B-1.27 to B-244.27, wherein the compound of the formula I is the compound 1.27 of table A and wherein the compound II is the compound given in one line of table B.

Particular embodiments of the invention are mixtures B-1.28 to B-244.28, wherein the compound of the formula I is the compound 1.28 of table A and wherein the compound II is the compound given in one line of table B.

Particular embodiments of the invention are mixtures B-1.29 to B-244.29, wherein the compound of the formula I is the compound 1.29 of table A and wherein the compound II is the compound given in one line of table B.

Particular embodiments of the invention are mixtures B-1.30 to B-244.30, wherein the compound of the formula I is the compound 1.30 of table A and wherein the compound II is the compound given in one line of table B.

Particular embodiments of the invention are mixtures B-1.31 to B-244.31, wherein the compound of the formula I is the compound 1.31 of table A and wherein the compound II is the compound given in one line of table B.

Particular embodiments of the invention are mixtures B-1.32 to B-244.32, wherein the compound of the formula I is the compound 1.32 of table A and wherein the compound II is the compound given in one line of table B.

Particular embodiments of the invention are mixtures B-1.33 to B-244.33, wherein the compound of the formula I is the compound 1.33 of table A and wherein the compound II is the compound given in one line of table B.

Particular embodiments of the invention are mixtures B-1.34 to B-244.34, wherein the compound of the formula I is the compound 1.34 of table A and wherein the compound II is the compound given in one line of table B.

Particular embodiments of the invention are mixtures B-1.35 to B-244.35, wherein the compound of the formula I is the compound 1.35 of table A and wherein the compound II is the compound given in one line of table B.

Particular embodiments of the invention are mixtures B-1.36 to B-244.36, wherein the compound of the formula I is the compound 1.36 of table A and wherein the compound II is the compound given in one line of table B.

Particular embodiments of the invention are mixtures B-1.37 to B-244.37, wherein the compound of the formula I is the compound 1.37 of table A and wherein the compound II is the compound given in one line of table B.

Particular embodiments of the invention are mixtures B-1.38 to B-244.38, wherein the compound of the formula I is the compound 1.38 of table A and wherein the compound II is the compound given in one line of table B.

Particular embodiments of the invention are mixtures B-1.39 to B-244.39, wherein the compound of the formula I is the compound 1.39 of table A and wherein the compound II is the compound given in one line of table B.

Particular embodiments of the invention are mixtures B-1.40 to B-244.40, wherein the compound of the formula I is the compound 1.40 of table A and wherein the compound II is the compound given in one line of table B.

Particular embodiments of the invention are mixtures B-1.41 to B-244.41, wherein the compound of the formula I is the compound 1.41 of table A and wherein the compound II is the compound given in one line of table B.

Particular embodiments of the invention are mixtures B-1.42 to B-244.42, wherein the compound of the formula I is the compound 1.42 of table A and wherein the compound II is the compound given in one line of table B.

Particular embodiments of the invention are mixtures B-1.43 to B-244.43, wherein the compound of the formula I is the compound 1.43 of table A and wherein the compound II is the compound given in one line of table B.

Particular embodiments of the invention are mixtures B-1.44 to B-244.44, wherein the compound of the formula I is the compound 1.44 of table A and wherein the compound II is the compound given in one line of table B.

Particular embodiments of the invention are mixtures B-1.45 to B-244.45, wherein the compound of the formula I is the compound 1.45 of table A and wherein the compound II is the compound given in one line of table B.

Particular embodiments of the invention are mixtures B-1.46 to B-244.46, wherein the compound of the formula I is the compound 1.46 of table A and wherein the compound II is the compound given in one line of table B.

Particular embodiments of the invention are mixtures B-1.47 to B-244.47, wherein the compound of the formula I is the compound 1.47 of table A and wherein the compound II is the compound given in one line of table B.

Particular embodiments of the invention are mixtures B-1.48 to B-244.48, wherein the compound of the formula I is the compound 1.48 of table A and wherein the compound II is the compound given in one line of table B.

Particular embodiments of the invention are mixtures B-1.49 to B-244.49, wherein the compound of the formula I is the compound 1.49 of table A and wherein the compound II is the compound given in one line of table B.

Particular embodiments of the invention are mixtures B-1.50 to B-244.50, wherein the compound of the formula I is the compound 1.50 of table A and wherein the compound II is the compound given in one line of table B.

Particular embodiments of the invention are mixtures B-1.51 to B-244.51, wherein the compound of the formula I is the compound 1.51 of table A and wherein the compound II is the compound given in one line of table B.

Particular embodiments of the invention are mixtures B-1.52 to B-244.52, wherein the compound of the formula I is the compound 1.52 of table A and wherein the compound II is the compound given in one line of table B.

Particular embodiments of the invention are mixtures B-1.53 to B-244.53, wherein the compound of the formula I is the compound 1.53 of table A and wherein the compound II is the compound given in one line of table B.

Particular embodiments of the invention are mixtures B-1.54 to B-244.54, wherein the compound of the formula I is the compound 1.54 of table A and wherein the compound II is the compound given in one line of table B.

Particular embodiments of the invention are mixtures B-1.55 to B-244.55, wherein the compound of the formula I is the compound 1.55 of table A and wherein the compound II is the compound given in one line of table B.

Particular embodiments of the invention are mixtures B-1.56 to B-244.56, wherein the compound of the formula I is the compound 1.56 of table A and wherein the compound II is the compound given in one line of table B.

Particular embodiments of the invention are mixtures B-1.57 to B-244.57, wherein the compound of the formula I is the compound 1.57 of table A and wherein the compound II is the compound given in one line of table B.

Particular embodiments of the invention are mixtures B-1.58 to B-244.58, wherein the compound of the formula I is the compound 1.58 of table A and wherein the compound II is the compound given in one line of table B.

Particular embodiments of the invention are mixtures B-1.59 to B-244.59, wherein the compound of the formula I is the compound 1.59 of table A and wherein the compound II is the compound given in one line of table B.

Particular embodiments of the invention are mixtures B-1.60 to B-244.60, wherein the compound of the formula I is the compound 1.60 of table A and wherein the compound II is the compound given in one line of table B.

Particular embodiments of the invention are mixtures B-1.61 to B-244.61, wherein the compound of the formula I is the compound 1.61 of table A and wherein the compound II is the compound given in one line of table B.

Particular preference is given to mixtures B-1.8 to B-243.8.

Particular preference is also given to mixtures B-1.16 to B-243.16.

For use according to the present invention, the mixtures according to the invention, or the compound I and the active compound II, can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner, for example by extending the active compound with solvents and/or carriers, if desired using emulsifiers and dispersants. Liquid carriers include aqueous and non-aqueous solvents. Liquid carriers include e.g. the solvents mentioned below.

Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
solid carriers such as ground natural minerals (for example kaolins, clays, talc, chalk) and ground synthetic minerals (for example highly disperse silica, silicates);
emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ethers, ethoxylated isooctylphenol, octylphenol, nonylphenol, alkylphenol polyglycol ethers, tributylphenyl polyglycol ethers, tristearylphenyl polyglycol ethers, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Also anti-freezing agents such as glycerin, ethylene glycol, propylene glycol and bactericides such as can be added to the formulation.

Suitable antifoaming agents are for example antifoaming agents based on silicon or magnesium stearate.

Seed treatment formulations may additionally comprise binders and optionally colorants.

Binders can be added to improve the adhesion of the active materials on the seeds after treatment. Suitable binders are block copolymers EO/PO surfactants but also polyvinylalcohols, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers, polyurethans, polyvinylacetate, tylose and copolymers derived from these polymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Example of a gelling agent is carrageen (Satiagel®).

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

For seed treatment purposes, respective formulations can be diluted 2-10 fold leading to concentrations in the ready to use preparations of 0,01 to 60% by weight active compound by weight, preferably 0,1 to 40% by weight.

The following are examples of formulations: 1. Products for dilution with water For seed treatment purposes, such products may be applied to the seed diluted or undiluted.

### A Water-Soluble Concentrates (SL, LS)

10 parts by weight of the active compounds are dissolved with 90 parts by weight of water or a water-soluble solvent. As an alternative, wetters or other auxiliaries are added. The active compound dissolves upon dilution with water. A formulation having an active compound content of 10% by weight is obtained in this manner.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight.

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added into 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants and wetters as well as silica gel. Dilution with water gives a stable dispersion or solution of the active compound. The active compound content of the formulation is 75% by weight.

### H Gel Formulations (GF)

In a ball mill, 20 parts by weight of the active compounds, 10 parts by weight of dispersant, 1 part by weight of gelling agent and 70 parts by weight of water or an organic solvent are ground to give a fine suspension. On dilution with water, a stable suspension having an active compound content of 20% by weight is obtained.

### 2. Products to be Applied Undiluted

I Dustable powders (DP, DS)

5 parts by weight of the active compounds are ground finely and mixed intimately with 95 parts by weight of finely divided kaolin. This gives a dustable product having an active compound content of 5% by weight.

### J Granules (GR, FG, GG, MG)

0.5 part by weight of the active compounds is ground finely and associated with 99.5 parts by weight of carriers. Current methods are extrusion, spray-drying or the fluidized bed. This gives granules to be applied undiluted having an active compound content of 0.5% by weight.

### K ULV Solutions (UL)

10 parts by weight of the active compounds are dissolved in 90 parts by weight of an organic solvent, for example xylene. This gives a product to be applied undiluted having an active compound content of 10% by weight.

For seed treatment, use is usually made of water-soluble concentrates (LS), suspensions (FS), dustable powders (DS), water-dispersible and water-soluble powders (WS, SS), emulsions (ES), emulsifiable concentrates (EC) and gel formulations (GF). These formulations can be applied to the seed in undiluted form or, preferably, diluted. Application can be carried out prior to sowing.

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

The inventive mixtures can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compounds without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:100 to 100:1, preferably 1:10 to 10:1.

Compositions of this invention may also contain fertilizers such as ammonium nitrate, urea, potash, and superphosphate, phytotoxicants and plant growth regulators and safeners. These may be used sequentially or in combination with the above-described compositions, if appropriate also added only immediately prior to use (tank mix). For example, the plant(s) may be sprayed with a composition of this invention either before or after being treated with the fertilizers.

The compounds can be applied simultaneously, that is jointly or separately, or in succession, the sequence, in the case of separate application, generally not having any effect on the result of the control measures.

Advantageously, they are suitable for controlling the following plant diseases:
Alternaria species on vegetables, oilseed rape, sugar beet and fruit and rice, such as, for example, A. solani or A. alternate on potatoes and tomatoes;
Aphanomyces species on sugar beet and vegetables;
Ascochyta species on cereals and vegetables;
Bipolaris and Drechslera species on corn, cereals, rice and lawns, such as, for example, D. maydis on corn;
Blumeria graminis (powdery mildew) on cereals;
Botrytis cinerea (gray mold) on strawberries, vegetables, flowers and grapevines;
Bremia lactucae on lettuce;
Cercospora species on corn, soybeans, rice and sugar beet;
Cochlibbolus species on corn, cereals, rice, such as, for example, Cochlibbolus sativus on cereals, Cochliobolus miyabeanus on rice;
Colletotricum species on soybeans and cotton;
Drechslera species, Pyrenophora species on corn, cereals, rice and lawns, such as, for example, D. teres on barley or D. tritici-repentis on wheat;
Esca on grapevines, caused by Phaeoacremonium chlamydosporium, Ph. Aleophilum and Formitipora punctata (syn. Phellinus punctatus);
Exserohllum species on corn;
Eryslphe cichoracearum and Sphaerotheca fuliginea on cucumbers;
Fusarium and Verticillium species on various plants, such as, for example, F. graminearum or F. culmorum on cereals or F. oxysporum on a multitude of plants, such as, for example, tomatoes;
Gaeumanomyces graminis on cereals;
Gibberella species on cereals and rice (for example Gibberella fujikuroi on rice);
Grainstaining complex on rice;
Helminthosporium species on corn and rice;
Michrodochium nivale on cereals;
Mycosphaerella species on cereals, bananas and groundnuts, such as, for example, M. graminicola on wheat or M. fijiensis on bananas;
Peronospora species on cabbage and bulbous plants, such as, for example, P. brassicae on cabbage or P. destructor on onions;
Phakopsara pachyrhizi and Phakopsara melbomiae on soybeans;
Phomopsis species on soybeans and sunflowers;
Phytophthora infestans on potatoes and tomatoes;
Phytophthora species on various plants, such as, for example, P. capsici on bell pepper;
Plasmopara viticola on grapevines;
Podosphaera leucotricha on apples;
Pseudocercosporella herpotrichoides on cereals;
Pseudoperonospora on various plants, such as, for example, P. cubensis on cucumber or P. humili on hops;
Puccinia species on various plants, such as, for example, P. triticina, P. striformins, P. hordei or P. graminis on cereals or P. asparagi on asparagus;
Pyricularia oryzae, Corticium sasakk, Sarocladium oryzae, S. attenuatum, Entyloma oryzae on rice;
Pyricularia grisea on lawns and cereals;
Pythium spp. on lawns, rice, corn, cotton, oilseed rape, sunflowers, sugar beet, vegetables and other plants, such as, for example, P. ultiumum on various plants, P. aphanidermatum on lawns;
Rhizoctonia species on cotton, rice, potatoes, lawns, corn, oilseed rape, potatoes, sugar beet, vegetables and on various plants, such as, for example, R. solani on beet and various plants;
Rhynchosporium secalis on barley, rye and triticale;
Sclerotinia species on oilseed rape and sunflowers;
Septoria tritici and Stagonospora nodorum on wheat;
Erysiphe (syn. Uncinula) necator on grapevines;
Setospaeria species on corn and lawns;
Sphacelotheca reilinia on corn;
Thievaliopsis species on soybeans and cotton;
Tilletia species on cereals;
Ustilago species on cereals, corn and sugar cane, such as, for example, U. maydis on corn;
Venturia species (scab) on apples and pears, such as, for example, V. inaequalis on apples.

The inventive mixtures are furthermore suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Pettiella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternana spp., Paecllomyces spp. and Zygomycetes, such as Mucor spp., additionally in the protection of materials the following yeasts: Candida spp. and Saccharomyces cerevisae.

They are particularly important for controlling a multitude of fungi on various cultivated plants, such as bananas, cotton, vegetable species (for example cucumbers, beans and cucurbits), barley, grass, oats, coffee, potatoes, corn, fruit species, rice, rye, soya, tomatoes, grapevines, wheat, ornamental plants, sugar cane and also on a large number of seeds.

“Locus” means a plant, seed, soil, area, material or environment in which a pest is growing or may grow.

In the mixtures according to the present invention, the at least one compound I and the at least one compound II are present in fungicidally effective amounts. In general, “fungicidally effective amount” means the amount of the inventive mixtures or of compositions comprising the mixtures needed to achieve an observable effect on growth, including the effects of necrosis, death, retardation, prevention, and removal, destruction, or otherwise diminishing the occurrence and activity of the target organism. The fungicidally effective amount can vary for the various mixtures/compositions used in the invention. A fungicidally effective amount of the mixtures/compositions will also vary according to the prevailing conditions such as desired fungicidal effect and duration, weather, target species, locus, mode of application, and the like.

The compound I and the compound II are usually applied in a weight ratio of from 500:1 to 1:100, preferably from 100:1 to 1:100, more preferably from 20:1 to 1:20, in particular from 10:1 to 1:10.

When preparing the mixtures, it is preferred to employ the pure active compounds I and II, to which further active compounds against pests, such as insects, arachnids or nematodes, or herbicidal or growth-regulating active compounds or fertilizers can be added as further active components according to need.

The inventive mixtures are employed by treating the fungi or the plants, seeds, materials or soil to be protected from fungal attack with a fungicidally effective amount of the active compounds. The application can be carried out both before and after the infection of the materials, plants or seeds by the fungi.

In the method of combating harmful fungi depending on the type of compound and the desired effect, the application rates of the mixtures according to the invention are from 5 g/ha to 2000 g/ha, preferably from 50 to 900 g/ha, in particular from 50 to 750 g/ha.

The inventive mixtures or compositions of these mixtures can also be employed for protecting plants from attack or infestation by insects, acarids or nematodes comprising contacting a plant, or soil or water in which the plant is growing.

In the context of the present invention, the term plant refers to an entire plant, a part of the plant or the propagation material of the plant, that is, the seed or the seedling.

The treatment can be made into the seedbox before planting into the field.

Plants which can be treated with the inventive mixtures include all genetically modified plants or transgenic plants, e.g. crops which tolerate the action of herbicides or fungicides or insecticides owing to breeding, including genetic engineering methods, or plants which have modified characteristics in comparison with existing plants, which can be generated for example by traditional breeding methods and/or the generation of mutants, or by recombinant procedures.

Some of the inventive mixtures have systemic action and can therefore be used for the protection of the plant shoot against foliar pests as well as for the treatment of the seed and roots against soil pests. The term seed treatment comprises all suitable seed treatment techniques known in the art, such as seed dressing, seed coating, seed dusting, seed soaking and seed pelleting.

The compound I and the compound II are usually applied in a weight ratio of from 500:1 to 1:100, preferably from 20:1 to 1:50, in particular from 5:1 to 1:20. Depending on the desired effect, the application rates of the mixtures according to the invention are from 5 g/ha to 2000 g/ha, preferably from 50 to 1500 g/ha, in particular from 50 to 750 g/ha.

The inventive mixtures are also suitable for the protection of the seed and the seedlings' roots and shoots, preferably the seeds, against soil pests.

Compositions, which are especially useful for seed treatment, are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)

### H Gel-Formulations (GF)

I Dustable powders (DP, DS)

Preferred are FS formulations.

In the treatment of seed, the application rates of the inventive mixture are generally from 0.001 to 10 kg per 100 kg of seed, dependent from the desired effect and the kind of seed. Application rates are preferably from 1 to 1000 g/100 kg of seed, more preferably from 1 to 750 g/100 kg, in particular from 5 to 500 g/100 kg. The separate or joint application of the compounds I and II or of the mixtures of the compounds I and II is carried out by spraying or dusting the seeds, the seedlings, the plants or the soils before or after sowing of the plants or before or after emergence of the plants.

The invention also relates to the propagation products of plants, and especially the seed comprising, that is, coated with and/or containing, a mixture as defined above or a composition containing the mixture of two or more active ingredients or a mixture of two or more compositions each providing one of the active ingredients. The seed comprises the inventive mixtures in an amount of from 0.1 g to 10 kg per 100 kg of seed.

The inventive mixtures are effective through both contact (via soil, glass, wall, bed net, carpet, plant parts or animal parts), and ingestion (bait, or plant part) and through trophallaxis and transfer.

Preferred application methods are into water bodies, via soil, cracks and crevices, pastures, manure piles, sewers, into water, on floor, wall, or by perimeter spray application and bait.

The inventive mixtures and the compositions comprising them can be used for protecting wooden materials such as trees, board fences, sleepers, etc. and buildings such as houses, outhouses, factories, but also construction materials, furniture, leathers, fibers, vinyl articles, electric wires and cables etc. from fungi.

Customary application rates in the protection of materials are, for example, from 0.01 g to 1000 g of active compound per m² treated material, desirably from 0.1 g to 50 g per m².

For use in spray compositions, the content of the mixture of the active ingredients is from 0.001 to 80 weights %, preferably from 0.01 to 50 weight % and most preferably from 0.01 to 15 weight %.

For use in treating crop plants, the rate of application of the mixture of the active ingredients of this invention may be in the range of 0.1 g to 4000 g per hectare, desirably from 25 g to 600 g per hectare, more desirably from 50 g to 500 g per hectare.

### BIOLOGICAL EXAMPLES

### Fungicidal Action

The fungicidal effect of the compound and the mixtures could be demonstrated by the following tests:

The active compounds, separately or jointly, were prepared as a stock solution comprising 0.25% by weight of active compound in acetone or DMSO. 1% by weight of the emulsifier Uniperol® EL (wetting agent having emulsifying and dispersant action based on ethoxylated alkylphenols) was added to this solution, and the mixture was diluted with water to the desired concentration.

The visually determined percentages of infected leaf areas were converted into efficacies in % of the untreated control:

The efficacy (E) is calculated as follows using Abbot's formula:

E=(1−α/β)·100

α corresponds to the fungicidal infection of the treated plants in % and
β corresponds to the fungicidal infection of the untreated (control) plants in %

An efficacy of 0 means that the infection level of the treated plants corresponds to that of the untreated control plants; an efficacy of 100 means that the treated plants were not infected.

The expected efficacies of mixtures of active compounds were determined using Colby's formula (Colby, S. R. “Calculating synergistic and antagonistic responses of herbicide Combinations”, Weeds, 15, 20-22, 1967) and compared with the observed efficacies.

Colby's formula:

E=x+y−x·y/100
E expected efficacy, expressed in % of the untreated control, when using the mixture of the active compounds A and B at the concentrations a and b
x efficacy, expressed in % of the untreated control, when using the active compound A at the concentration a
y efficacy, expressed in % of the untreated control, when using the active compound B at the concentration b

### Use Example 1

### Fungicidal Control of Brown Spot Caused by Cochliobolus miyabeanus (protective)

Leaves of pot-grown rice seedlings were sprayed to run-off with an aqueous suspension, containing the concentration of active ingredient as described below prepared from the stock solution. The plants were allowed to air-dry. At the following day the plants were inoculated with an aqueous spore suspension of Cochliobolus miyabeanus. Then the trial plants were immediately transferred to a humid chamber. After 6 days at 22-24° C. and a relative humidity close to 100% the extent of fungal attack on the leaves was visually assessed as % diseased leaf area.

Microtest (Use examples 2, 3 and 4)

The active compounds were formulated separately as a stock solution having a concentration of 10000 ppm in dimethyl sulfoxide.

### Use Example 2

### Activity Against the Late Blight Pathogen Phytophthora infestans in the Microtiter Test (Phytin)

The stock solutions were mixed according to the ratio, pipetted onto a microtiter plate (MTP) and diluted with water to the stated concentrations. A spore suspension of Phytophtora infestans containing a pea juice-based aqueous nutrient medium was then added. The plates were placed in a water vapor-saturated chamber at a temperature of 18° C. Using an absorption photometer, the MTPs were measured at 405 nm 7 days after the inoculation. The measured parameters were compared to the growth of the active compound-free control variant (100%) and the fungus-free and active compound-free blank value to determine the relative growth in % of the pathogens in the respective active compounds. These percentages were converted into efficacies. An efficacy of 0 means that the growth level of the pathogens corresponds to that of the untreated control; an efficacy of 100 means that the pathogens were not growing. The test results are shown in Tables 1 and 2.

The compounds of the formula I used were compounds 1.8 and 1.16.

The fungicidal compound II was selected from
azoles: prothioconazole;
strobilurins: dimoxystrobin;
heterocyclic compounds: 5-ethyl-6-octyl-[1,2,4]triazolo[1,5-a]pyrimidine-7-ylamine, captan;
carbamates: iprovalicarb;
other active compounds: chlorothalonil.

TABLE 1

| | | | | Calculated | |
|---|---|---|---|---|---|
| | | | Observed | efficacy | |
| Active compound/ | Concentration | | efficacy | according to | Synergism |
| active mixture | (ppm) | Mixture | (%) | Colby (%) | (%) |
| I.8 | 4 | — | 19 | | |
| | 1 | — | 4 | | |
| | 0.25 | — | 1 | | |
| | 0.063 | — | 2 | | |
| Dimoxystrobin | 0.25 | — | 42 | | |
| 5-ethyl-6-octyl- | 0.25 | — | 29 | | |
| [1,2,4]triazolo[1,5-a]pyrimidine- | | | | | |
| 7-ylamine | | | | | |
| Prothioconazole | 16 | — | 22 | | |
| Iprovalicarb | 1 | — | 66 | | |
| Chlorothalonil | 0.25 | — | 10 | | |
| 1.8 | 1 | 4:1 | 64 | 44 | 20 |
| Dimoxystrobin | 0.25 | | | | |
| 1.8 | 0.063 | 1:4 | 57 | 31 | 26 |
| 5-ethyl-6-octyl- | 0.25 | | | | |
| [1,2,4]triazolo[1,5-a]pyrimidine- | | | | | |
| 7-ylamine | | | | | |
| 1.8 | 4 | 1:4 | 81 | 37 | 44 |
| Prothioconazol | 16 | | | | |
| 1.8 | 1 | 1:1 | 96 | 67 | 29 |
| Iprovalicarb | 1 | | | | |
| 1.8 | 0.25 | 1:1 | 43 | 11 | 32 |
| Chlorothalonil | 0.25 | | | | |

TABLE 2

| | | | | Calculated | |
|---|---|---|---|---|---|
| | | | Observed | efficacy | |
| Active compound/active | Concentration | | efficacy | according to | Synergism |
| mixture | (ppm) | Mixture | (%) | Colby (%) | (%) |
| I.16 | 1 | — | 0 | | |
| Captan | 1 | — | 26 | | |
| 1.16 | 1 | 1:1 | 95 | 26 | 69 |
| Captan | 1 | | | | |

### Use example 3

### Activity Against Rice Blast Pyricularia oryzae in the Microtiterplate Test (Pyrior)

The stock solutions were mixed according to the ratio, pipetted onto a micro titer plate (MTP) and diluted with water to the stated concentrations. A spore suspension of Pyricularia oryzae in an aqueous biomalt solution was then added. The plates were placed in a water vapor-saturated chamber at a temperature of 18° C. Using an absorption photometer, the MTPs were measured at 405 nm 7 days after the inoculation. The measured parameters were compared to the growth of the active compound-free control variant (100%) and the fungus-free and active compound-free blank value to determine the relative growth in % of the pathogens in the respective active compounds. These percentages were converted into efficacies. An efficacy of 0 means that the growth level of the pathogens corresponds to that of the untreated control; an efficacy of 100 means that the pathogens were not growing. The results are shown in Tables 3 and 4.

The compounds of the formula I used were compounds 1.8 and 1.16.

The fungicidal compound II was selected from
azoles: carbendazim;
carbamates: propineb.

TABLE 3

| | | | | Calculated | |
|---|---|---|---|---|---|
| | | | Observed | efficacy | |
| Active compound/ | Concentration | | efficacy | according to | Synergism |
| active mixture | (ppm) | Mixture | (%) | Colby (%) | (%) |
| I.8 | 1 | — | 0 | | |
| | 0.25 | — | 2 | | |
| Carbendazim | 0.25 | — | 24 | | |
| I.8 | 0.25 | 1:1 | 48 | 25 | 23 |
| Carbendazim | 0.25 | | | | |

TABLE 4

| | | | | Calculated | |
|---|---|---|---|---|---|
| | | | Observed | efficacy | |
| Active compound/ | Concentration | | efficacy | according to | Synergism |
| active mixture | (ppm) | Mixture | (%) | Colby (%) | (%) |
| I.16 | 1 | — | 0 | | |
| Propineb | 1 | — | 23 | | |
| 1.16 | 1 | 1:1 | 46 | 23 | 23 |
| Propineb | 1 | | | | |

### Use Example 4

### Activity Against Leaf Blotch on Wheat Caused by Septoria tritici in the Microtiter Test (Septtr)

The stock solution were mixed according to the ratio, pipetted onto a microtiter plate (MTP) and diluted with water to the stated concentrations. A spore suspension of Septoria tritici in an aqueous biomalt solution was then added. The plates were placed in a water vapor-saturated chamber at temperatures of 18° C. Using an absorption photometer, the microtiter plates were measured at 405 nm on day 7 after the inoculation. The measured parameters were compared to the growth of the active compound-free control variant (100%) and the fungus-free and active compound-free blank value to determine the relative growth in % of the pathogens in the respective active compounds. These percentages were converted into efficacies. An efficacy of 0 means that the growth level of the pathogens corresponds to that of the untreated control; an efficacy of 100 means that the pathogens were not growing. The test results are shown in Table 5.

The compound of the formula I used was compound 1.8.

The fungicidal compound II was selected from
azoles: tebuconazole;
heterocyclic compounds: fenpropimorph, tridemorph.

TABLE 5

| | | | | Calculated | |
|---|---|---|---|---|---|
| | | | Observed | efficacy | |
| Active compound/ | Concentration | | efficacy | according to | Synergism |
| active mixture | (ppm) | Mixture | (%) | Colby (%) | (%) |
| I.8 | 0.25 | — | 10 | | |
| | 0.063 | — | 4 | | |
| Fenpropimorph | 1 | — | 9 | | |
| Tridemorph | 1 | — | 3 | | |
| Tebuconazol | 0.25 | — | 63 | | |
| I.8 | 0.25 | 1:4 | 69 | 19 | 50 |
| Fenpropimorph | 1 | | | | |
| I.8 | 0.25 | 1:4 | 75 | 13 | 62 |
| Tridemorph | 1 | | | | |
| I.8 | 0.063 | 1:4 | 85 | 64 | 21 |
| Tebuconazole | 0.25 | | | | |

The test results show that, by virtue of strong synergism, the activity of the mixtures according to the invention is considerably higher than had been predicted using Colby's formula.

1-30. (canceled)

31. A fungicidal mixture comprising, as active components,
1) a fungicidal compound of formula I 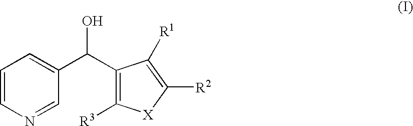
wherein:
X is O, S or NR⁴;
R¹ is selected from the group consisting of C₁-C₆-alkyl, C₁-C₆-alkoxy-C₁-C₆-alkyl, C₁-C₆-haloalkyl, aryl-C₁-C₄-alkyl, aryloxy-C₁-C₄-alkyl, arylthio-C₁-C₄-alkyl, aryl and heteroaryl, wherein the cyclic moieties of the last five radicals are unsubstituted or substituted with 1, 2 or 3 radicals selected from the group consisting of halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro;
R² is H or C₁-C₄-alkyl;
R³ is C₁-C₆-alkyl, C₁-C₆-alkoxy-C₁-C₆-alkyl, C₁-C₆-haloalkyl, aryl-C₁-C₄-alkyl, aryloxy-C₁-C₄-alkyl, arylthio-C₁-C₄-alkyl, aryl or heteroaryl, wherein the cyclic moieties of the last five radicals are unsubstituted or substituted with 1, 2 or 3 radicals selected from the group consisting of halogen, C₁-C₄-alkyl, C₂-C₄-alkenyl, C₂-C₄-alkynyl, C₁-C₄-alkoxy, C₁-C₄-alkylthio, C₁-C₄-haloalkoxy, C₁-C₄-haloalkylthio, cyano and nitro;
R⁴ is H, C₁-C₆-alkyl, C₁-C₆-alkoxy-C₁-C₆-alkyl, C₁-C₆-haloalkyl, aryl-C₁-C₄-alkyl, aryloxy-C₁-C₄-alkyl, arylthio-C₁-C₄-alkyl, aryl or heteroaryl, wherein the cyclic moieties of the last five radicals are unsubstituted or substituted with 1, 2 or 3 radicals selected from the group consisting of halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro;
or a salt thereof;
and
2) a fungicidal compound II selected from:
A) an azole, selected from the group consisting of azaconazole, diniconazole-M, oxpoconazol, uniconazole, 1-(4-chloro-phenyl)-2-([1,2,4]triazol-1-yl)-cycloheptanol, imazalil-sulfphate, bitertanole, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, paclobutrazol, penconazole, propiconazole, prothioconazole, simeconazole, tebuconazole, tetraconazole, uniconazole-P, triadimenol, triadimefon, triticonazole, cyazofamid, imazalil, pefurazoate, prochloraz, triflumizol, benomyl, carbendazim, fuberidazole, thiabendazole, ethaboxam, etridiazole, hymexazole and 1-(4-chloro-phenyl)-1-(propyn-2-yloxy)-3-(4-(3,4-dimethoxy-phenyl)-isoxazole-5-yl)-propane-2-one;
B) a strobilurin, selected from the group consisting of 2-(2-(6-(3-chloro-2-methylphenoxy)-5-fluoro-pyrimidin-4-yloxy)-phenyl)-2-methoxyimino-N-methylacetamide, 3-methoxy-2-(2-(N-(4-methoxy-phenyl)-cyclopropane-carboximidoylsulfanylmethyl)-phenyl)-acrylic acid methyl ester, azoxystrobin, dimoxystrobin, enestroburin, fluoxastrobin, kresoxim-methyl, metominostrobin, orysastrobin, picoxystrobin, pyraclostrobin, trifloxystrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, and methyl 2-(ortho-((2,5-dimethylphenyloxymethylene)phenyl)-3-methoxyacrylate;
C) a carboxamide, selected from the group consisting of benalaxyl-M, 2-amino-4-methyl-thiazole-5-carboxylic acid anilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxylic acid amide, N-(4′-chloro-3′,5-difluoro-biphenyl-2-yl)-3-difluoromethyl-1-methyl-1H-pyrazole-4-carboxylic acid amide, N-(4′-chloro-3′,5-difluoro-biphenyl-2-yl)-3-trifluoromethyl-1-methyl-1H-pyrazole-4-carboxylic acid amide, N-(3′,4′-dichloro-5-fluorobiphenyl-2-yl)-3-trifluoromethyl-1-methyl-1H-pyrazole-4-carboxylic acid amide, N-(3′,5-difluoro-4′-methyl-biphenyl-2-yl)-3-difluoromethyl-1-methyl-1H-pyrazole-4-carboxylic acid amide, N-(3′,5-difluoro-4′-methyl-biphenyl-2-yl)-3-trifluoromethyl-1-methyl-1H-pyrazole-4-carboxylic acid amide, N-(cis-2-bicyclopropyl-2-yl-phenyl)-3-di-fluoromethyl-1-methyl-1H-pyrazole-4-carboxylic acid amide, N-(trans-2-bicyclopropyl-2-yl-phenyl)-3-difluoromethyl-1-methyl-1H-pyrazole-4-carboxylic acid amide, fluopyram, N-(3-ethyl-3,5,5-trimethyl-cyclohexyl)-3-formylamino-2-hydroxy-benzamide, oxytetracyclin, silthiofam, N-(6-methoxy-pyridin-3-yl)cyclopropanecarboxylic acid amide, carboxin, benalaxyl, boscalid, fenhexamid, flutolanil, furametpyr, mepronil, metalaxyl, mefenoxam, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(3′,4′-dichloro-5-fluorobiphenyl-2-yl)-3-difluoromethyl-1-methylpyrazole-4-carboxamide, 3,4-dichloro-N-(2-cyanophenyl)isothiazol-5-carboxamide, N-(2′,4′-difluorobiphenyl-2-yl)-1-methyl-3-trifluoromethyl-1H-pyrazole-4-carboxamide, N-(2′,4′-dichloro-biphenyl-2-yl)-1-methyl-3-trifluoromethyl-1H-pyrazole-4-carboxamide; N-(2′,4′-difluorobiphenyl-2-yl)-3-difluoromethyl-1-methyl-1H-pyrazole-4-carboxamide; N-(2′,4′-dichlorobiphenyl-2-yl)-3-difluoromethyl-1-methyl-1H-pyrazole-4-carboxamide, N-(2′,5′-difluorobiphenyl-2-yl)-1-methyl-3-trifluoromethyl-1H-pyrazole-4-carboxamide, N-(2′,5′-dichlorobiphenyl-2-yl)-1-methyl-3-trifluoromethyl-1H-pyrazole-4-carboxamide, N-(2′,5′-difluorobiphenyl-2-yl)-3-difluoromethyl-1-methyl-1H-pyrazole-4-carboxamide, N-(2′,5′-dichlorobiphenyl-2-yl)-3-difluoromethyl-1-methyl-1H-pyrazole-4-carboxamide, N-(3′,5′-difluorobiphenyl-2-yl)-1-methyl-3-trifluoromethyl-1H-pyrazole-4-carboxamide; N-(3′,5′-dichlorobiphenyl-2-yl)-1-methyl-3-trifluoromethyl-1H-pyrazole-4-carboxamide, N-(3′,5′-difluorobiphenyl-2-yl)-3-difluoromethyl-1-methyl-1H-pyrazole-4-carboxamide, N-(3′,5′-dichlorobiphenyl-2-yl)-3-difluoromethyl-1-methyl-1H-pyrazole-4-carboxamide, N-(3′-fluorobiphenyl-2-yl)-1-methyl-3-trifluoromethyl-1H-pyrazole-4-carboxamide, N-(3′-chlorobiphenyl-2-yl)-1-methyl-3-trifluoromethyl-1H-pyrazole-4-carboxamide, N-(3′-fluorobiphenyl-2-yl)-3-difluoromethyl-1-methyl-1H-pyrazole-4-carboxamide, N-(3′-chlorobiphenyl-2-yl)-3-difluoromethyl-1-methyl-1H-pyrazole-4-carboxamide, N-(T-fluorobiphenyl-2-yl)-1-methyl-3-trifluoromethyl-1H-pyrazole-4-carboxamide, N-(2′-chlorobiphenyl-2-yl)-1-methyl-3-trifluoromethyl-1H-pyrazole-4-carboxamide, N-(2′-fluorobiphenyl-2-yl)-3-difluoromethyl-1-methyl-1H-pyrazole-4-carboxamide, N-(2′-chlorbiphenyl-2-yl)-3-difluoromethyl-1-methyl-1H-pyrazole-4-carboxamide, N-(2′-fluoro-4′-chloro-5′-methylbiphenyl-2-yl)-1-methyl-3-trifluoromethyl-1H-pyrazole-4-carboxamide, N-(3′,4′,5′-trifluorobiphenyl-2-yl)-1-methyl-3-trifluoromethyl-1H-pyrazole-4-carboxamide, N-(3′,4′,5′-trifluorobiphenyl-2-yl)-1-methyl-3-difluoromethyl-1H-pyrazole-4-carboxamide; N-(2′,4′,5′-trifluorobiphenyl-2-yl)-1-methyl-3-difluoromethyl-1H-pyrazole-4-carboxamide, N-(3′,4′,5′-trifluorobiphenyl-2-yl)-3-chlorofluoromethyl-1-methyl-1H-pyrazole-4-carboxamide, N-[2-(1,1,2,3,3,3-hexafluoropropoxy)phenyl]-1-methyl-3-trifluoromethyl-1H-pyrazole-4-carboxamide; N-[2-(1,1,2,3,3,3-hexafluoropropoxy)-phenyl]-3-difluoromethyl-1-methyl-1H-pyrazole-4-carboxamide, N-[2-(2-chloro-1,1,2-trifluoroethoxy)phenyl]-1-methyl-3-trifluoromethyl-1H-pyrazole-4-carboxamide; N-[2-(2-chlor-1,1,2-trifluoroethoxy)phenyl]-3-difluoromethyl-1-methyl-1H-pyrazole-4-carboxamide, N-[2-(1,1,2,2-tetrafluoroethoxy)phenyl]-3-difluoromethyl-1-methyl-1H-pyrazole-4-carboxamide, N-[2-(1,1,2,2-tetrafluoroethoxy)phenyl]-1-methyl-3-trifluoromethyl-1H-pyrazole-4-carboxamide; N-(4′-(trifluoromethylthio)biphenyl-2-yl)-3-difluoromethyl-1-methyl-1H-pyrazole-4-carboxamide, N-(4′-(trifluoromethylthio)biphenyl-2-yl)-1-methyl-3-trifluoromethyl-1H-pyrazole-4-carboxamide, dimethomorph, flumorph, flumetover, fluopicolide (picobenzamid), zoxamide, carpropamid, diclocymet, mandipropamid, N-(2-{4-[3-(4-chlorophenyl)prop-2-ynyloxy]-3-methoxyphenyl}ethyl)-2-methanesulfonylamino-3-methylbutyramide, and N-(2-{4-[3-(4-chlorophenyl)-prop-2-ynyloxy]-3-methoxyphenyl}ethyl)-2-ethanesulfonylamino-3-methylbutyramide;
D) a heterocyclic compound, selected from the group consisting of 2,3,5,6-tetrachloro-4-methanesulfonyl-pyridine, 3,4,5-trichloropyridine-2,6-di-carbonitrile, N-(1-(5-bromo-3-chloro-pyridin-2-yl)-ethyl)-2,4-dichloronicotinamide, N-[(5-bromo-3-chloro-pyridin-2-yl)-methyl]-2,4-dichloro-nicotinamide, diflumetorim, nitrapyrin, dodemorph acetate, fluoroimid, blasticidin-S, chinomethionat, debacarb, difenzoquat, difenzoquat-methylsulphat, oxolinic acid, piperalin, fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, probenazole, amisulbrom, anilazine, diclomezine, pyroquilon, proquinazid, tricyclazole, 2-butoxy-6-iodo-3-propylchromen-4-one, acibenzolar-5-methyl, captafol, captan, dazomet, folpet, fenoxanil, quinoxyfen, 3-[5-(4-chlorophenyl)-2,3-dimethylisoxazolidin-3-yl]pyridine, 5-chloro-7-(4-methylpiperidin-1-yl)-6-(2,4,6-trifluorophenyl)-[1,2,4]triazolo[1,5-a]pyrimidine, 6-(3,4-dichloro-phenyl)-5-methyl-[1,2,4]triazolo[1,5-a]pyrimidine-7-ylamine, 6-(4-tert-butylphenyl)-5-methyl-[1,2,4]triazolo[1,5-a]pyrimidine-7-ylamine, 5-methyl-6-(3,5,5-trimethyl-hexyl)-[1,2,4]triazolo[1,5-a]pyrimidine-7-ylamine, 5-methyl-6-octyl-[1,2,4]triazolo[1,5-a]pyrimidine-7-ylamine, 6-methyl-5-octyl-[1,2,4]triazolo[1,5-a]pyrimidine-7-ylamine, 6-ethyl-5-octyl-[1,2,4]triazolo[1,5-a]pyrimidine-7-ylamine, 5-ethyl-6-octyl-[1,2,4]triazolo[1,5-a]pyrimidine-7-ylamine, 5-ethyl-6-(3,5,5-trimethyl-hexyl)-[1,2,4]triazolo[1,5-a]pyrimidine-7-ylamine, 6-octyl-5-propyl-[1,2,4]triazolo[1,5-a]pyrimidine-7-ylamine, 5-methoxymethyl-6-octyl-[1,2,4]triazolo[1,5-a]pyrimidine-7-ylamine, 6-octyl-5-trifluoromethyl-[1,2,4]triazolo[1,5-a]pyrimidine-7-ylamine and 5-trifluoromethyl-6-(3,5,5-trimethyl-hexyl)-[1,2,4]triazolo[1,5-a]pyrimidine-7-ylamine;
E) a carbamate, selected from the group consisting of methasulphocarb, propamocarb hydrochlorid, mancozeb, maneb, metam, metiram, ferbam, propineb, thiram, zineb, ziram, thiophanate-methyl, diethofencarb, iprovalicarb, flubenthiavalicarb, propamocarb, methyl 3-(4-chlorophenyl)-3-(2-isopropoxycarbonylamino-3-methylbutyrylamino)propanoate, and 4-fluorophenyl N-(1-(1-(4-cyanophenyl)ethanesulfonyl)-but-2-yl)carbamate;
and
F) another active compound, selected from the group consisting of dodine, iminoctadine, guazatine, dodine free base, guazatine-acetate, iminoctadine-triacetate, kasugamycin, streptomycin, polyoxine, validamycin A, binapacryl, dinocap, dinobuton, dithianon, isoprothiolane, fentin acetate, edifenphos, iprobenfos, fosetyl, fosetyl-aluminum, phosphorous acid and its salts, pyrazophos, tolclofos-methyl, chlorothalonil, dichlofluanid, flusulfamide, hexachlorobenzene, phthalide, pencycuron, quintozene, tolylfluanid, Bordeaux mixture, copper acetate, copper hydroxide, copper oxychloride, basic copper sulfate, sulfur, cyflufenamid, cymoxanil, dimethirimol, ethirimol, furalaxyl, metrafenone, spiroxamine, iminoctadine-tris(albesilate), kasugamycin-hydrochlorid-hydrat, dichlorophen, pentachlorophenol and its salts, N-(4-chloro-2-nitro-phenyl)-N-ethyl-4-methylbenzenesulfonamide, dicloran, nitrothal-isopropyl, tecnazen, biphenyl, bronopol, diphenylamine, mildiomycin, oxin-copper, prohexadione calcium, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethylphenyl)-N-ethyl-N-methyl formamidine, N-(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine and N′-(5-difluoromethyl-2-methyl-4-(3-trimethylsilanyl-propoxy)-phenyl)-N-ethyl-N-methyl formamidine;
in synergistically effective amounts.

32. The mixture of claim 31, wherein R¹ is phenyl, thienyl or furyl, wherein the cyclic moieties of the radicals are unsubstituted or substituted with 1, 2 or 3 radicals selected from the group consisting of a halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro.

33. The mixture of claim 31, wherein R¹ is selected from the group consisting of 2-chlorophenyl, 3-chlorophenyl, 4-chlorophenyl, 2,4-dichlorophenyl, 2-chloro-4-fluorophenyl, 2-fluorophenyl, 3-fluorophenyl, 4-fluorophenyl, 2,4-difluorophenyl, 2,5-difluorophenyl, 3,5-difluorophenyl, 3-chloro-4-fluorophenyl, 4-chloro-2-fluorophenyl, 2-chloro-5-trifluoromethylphenyl, 2-chloro-4-trifluoromethylphenyl, 2-trifluoromethylphenyl, 3-trifluoromethylphenyl, 4-trifluoromethylphenyl, 4-methoxyphenyl, 4-trifluoromethoxyphenyl, 2-thienyl, 5-chloro-2-thienyl, 5-methyl-2-thienyl, 3-thienyl, 2-furyl and 5-chloro-2-furyl.

34. The mixture of claim 31, wherein R² is hydrogen.

35. The mixture of claim 31, wherein R³ is phenyl or thienyl, wherein the cyclic moieties of the 2 mentioned radicals are unsubstituted or substituted with 1, 2 or 3 radicals selected from the group consisting of halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro.

36. The mixture of claim 31, wherein R³ is selected from the group consisting of 2-chlorophenyl, 3-chlorophenyl, 4-chlorophenyl, 2,4-dichlorophenyl, 2-chloro-4-fluorophenyl, 2-fluorophenyl, 3-fluorophenyl, 4-fluorophenyl, 2,4-difluorophenyl, 2,5-difluorophenyl, 3,5-difluorophenyl, 3-chloro-4-fluorophenyl, 4-chloro-2-fluorophenyl, 2-chloro-5-trifluoromethylphenyl, 2-chloro-4-trifluoromethylphenyl, 2-trifluoromethylphenyl, 3-trifluoromethylphenyl, 4-trifluoromethylphenyl, 4-methoxyphenyl, 4-trifluoromethoxyphenyl, 2-thienyl, 5-chloro-2-thienyl, 5-bromo-2-thienyl, 5-methyl-2-thienyl and 3-thienyl.

37. The mixture of claim 31, wherein X is S.

38. The mixture of claim 31, wherein X is O.

39. The mixture of claim 31, wherein X is N—CH₃.

40. The mixture of claim 31, wherein:
R¹ is phenyl, thienyl or furyl, wherein the cyclic moieties of these radicals are unsubstituted or substituted with 1, 2 or 3 radicals selected from the group consisting of halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro;
R₂ is hydrogen;
R³ is phenyl or thienyl, wherein the cyclic moieties of these radicals are unsubstituted or substituted with 1, 2 or 3 radicals selected from halogen, C₁-C₄-alkyl, C₂-C₄-alkenyl, C₂-C₄-alkynyl, C₁-C₄-haloalkyl, C₁-C₄-alkoxy, C₁-C₄-alkylthio, C₁-C₄-haloalkoxy, C₁-C₄-haloalkylthio, cyano and nitro, and
X is S;
or a salt thereof.

41. The mixture of claim 31, wherein:
R¹ is selected from the group consisting of 2-chlorophenyl, 3-chlorophenyl, 4-chlorophenyl, 2,4-dichlorophenyl, 2-chloro-4-fluorophenyl, 2-fluorophenyl, 3-fluorophenyl, 4-fluorophenyl, 2,4-difluorophenyl, 2,5-difluorophenyl, 3,5-difluorophenyl, 3-chloro-4-fluorophenyl, 4-chloro-2-fluorophenyl, 2-chloro-5-trifluoromethylphenyl, 2-chloro-4-trifluoromethylphenyl, 2-trifluoromethylphenyl, 3-trifluoromethylphenyl, 4-trifluoromethylphenyl, 4-methoxyphenyl, 4-trifluoromethoxyphenyl, 2-thienyl, 5-chloro-2-thienyl, 5-methyl-2-thienyl, 3-thienyl, 2-furyl and 5-chloro-2-furyl;
R₂ is hydrogen;
R³ is selected from the group consisting of tert.-butyl, 2-chlorophenyl, 3-chlorophenyl, 4-chlorophenyl, 2,4-dichlorophenyl, 2-chloro-4-fluorophenyl, 2-fluorophenyl, 3-fluorophenyl, 4-fluorophenyl, 2,4-difluorophenyl, 2,5-difluorophenyl, 3,5-difluorophenyl, 3-chloro-4-fluorophenyl, 4-chloro-2-fluorophenyl, 2-chloro-5-trifluoromethylphenyl, 2-chloro-4-trifluoromethylphenyl, 2-trifluoromethylphenyl, 3-trifluoromethylphenyl, 4-trifluoromethylphenyl, 4-methoxyphenyl, 4-trifluoromethoxyphenyl, 2-thienyl, 5-chloro-2-thienyl, 5-methyl-2-thienyl, 5-bromo-2-thienyl and 3-thienyl; and
X is S;
or a salt thereof.

42. The mixture of claim 31, wherein the compound of formula I is selected from the group consisting of:
2,4-Bis-(3-chlorophenyl)-3-[(3-pyridyl)hydroxymethyl]thiophene, 4-(3-Chlorophenyl)-2-(5-chloro-2-thienyl)-3-[(3-pyridyl)hydroxymethyl]thiophene, 4-(3-Chlorophenyl)-2-(3,5-difluorophenyl)-3-[(3-pyridyl)hydroxymethyl]thiophene, 4-(4-Chlorophenyl)-2-(5-chloro-2-thienyl)-3-[(3-pyridyl)hydroxymethyl]thiophene, 4-(4-Chlorophenyl)-2-(3,5-difluorophenyl)-3-[(3-pyridyl)hydroxymethyl]thiophene, 2-(4-Chlorophenyl)-4-(2,4-difluorophenyl)-3-[(3-pyridyl)hydroxymethyl]thiophene, 4-(2,4-Difluorophenyl)-2-(1,1-dimethylethyl)-3-[(3-pyridyl)hydroxymethyl]thiophene, 2,4-Bis-(4-chlorophenyl)-3-[(3-pyridyl)hydroxymethyl]thiophene, 4-(4-Chlorophenyl)-3-[(3-pyridyl)hydroxymethyl]-2-(2-thienyl)thiophene, 2-(4-Chlorophenyl)-4-(5-chloro-2-thienyl)-3-[(3-pyridyl)hydroxymethyl]thiophene, 4-(5-Chloro-2-thienyl)-2-(2,4-difluorophenyl)-3-[(3-pyridyl)hydroxymethyl]thiophene, 2-(4-Chlorophenyl)-3-[(3-pyridyl)hydroxymethyl]-4-(2-thienyl)thiophene, 2-(2,4-Difluorophenyl)-3-[(3-pyridyl)hydroxymethyl]-4-(2-thienyl)thiophene, 2-(2,4-Difluorophenyl)-3-[(3-pyridyl)hydroxymethyl]-4-(2-thienyl)thiophene, 2-(4-Butylphenyl)-4-(5-methyl-2-thienyl)-3-[(3-pyridyl)hydroxymethyl]thiophene, 2,4-Bis-(2,4-Difluorophenyl)-3-[(3-pyridyl)hydroxymethyl]thiophene, 4-(4-Chlorophenyl)-2-(2,4-difluorophenyl)-3-[(3-pyridyl)hydroxymethyl]thiophene, 2,4-Bis-(2-trifluoromethylphenyl)-3-[(3-pyridyl)hydroxymethyl]thiophene, 2,4-Bis-(3-trifluoromethylphenyl)-3-[(3-pyridyl)hydroxymethyl]thiophene, 2,4-Bis-(4-trifluoromethylphenyl)-3-[(3-pyridyl)hydroxymethyl]thiophene, 4-(4-Chlorophenyl)-3-[(3-pyridyl)hydroxymethyl]-2-(3-thienyl)thiophene, 2-(5-Bromo-2-thienyl)-4-(4-chlorophenyl)-3-[(3-pyridyl)hydroxymethyl]thiophene, 4-(4-Chlorophenyl)-2-(5-methyl-2-thienyl)-3-[(3-pyridyl)hydroxymethyl]thiophene, 2-(3,5-Difluorophenyl)-3-[(3-pyridyl)hydroxymethyl]-4-(3-thienyl)thiophene, 2-(2,4-Difluorophenyl)-3-[(3-pyridyl)hydroxymethyl]-4-(3-thienyl)thiophene, 2-(3,5-Difluorophenyl)-4-(4-fluorophenyl)-3-[(3-pyridyl)hydroxymethyl]thiophene, 2-(2,4-Difluorophenyl)-4-(4-fluorophenyl)-3-[(3-pyridyl)hydroxymethyl]thiophene, 2-(4-Chlorophenyl)-3-[(3-pyridyl)hydroxymethyl]-4-(3-thienyl)thiophene, 4-(2,4-Difluorophenyl)-3-[(3-pyridyl)hydroxymethyl]-2-(3-thienyl)thiophene, 4-(2,4-Difluorophenyl)-3-[(3-pyridyl)hydroxymethyl]-2-(2-thienyl)thiophene, 2-(2,4-Difluorophenyl)-4-(2-fluorophenyl)-3-[(3-pyridyl)hydroxymethyl]thiophene, 2,4-Bis-(2-chlorophenyl)-3-[(3-pyridyl)hydroxymethyl]-thiophene, 2,4-Bis-(3-chlorophenyl)-3-[(3-pyridyl)hydroxymethyl]thiophene, 2,4-Bis-(phenyl)-3-[(3-pyridyl)hydroxymethyl]thiophene, 2,4-Bis-(2,4-Dichlorophenyl)-3-[(3-pyridyl)hydroxymethyl]thiophene, 2,4-Bis-(2-fluorophenyl)-3-[(3-pyridyl)hydroxymethyl]thiophene, 2,4-Bis-(3-fluorophenyl)-3-[(3-pyridyl)hydroxymethyl]thiophene, 2-(3-Chlorophenyl)-4,5-dimethyl-3-[(3-pyridyl)hydroxymethyl]thiophene, 4-(5-Chloro-2-furanyl)-2-(4-chlorophenyl)-3-[(3-pyridyl)hydroxymethyl]thiophene, 4-(5-Chloro-2-furanyl)-2-(2,4-difluorophenyl)-3-[(3-pyridyl)hydroxymethyl]-thiophene, 2,4-Bis-(2-thienyl)-3-[(3-pyridyl)hydroxymethyl]thiophene, 2,4-Bis-(4-fluorophenyl)-3-[(3-pyridyl)hydroxymethyl]thiophene, 2-(3-Chlorophenyl)-4-phenyl-3-[(3-pyridyl)hydroxymethyl]thiophene, 2,4-Bis-(3-chloro-5-trifluoromethylphenyl)-3-[(3-pyridyl)hydroxymethyl]-thiophene, 2,4-Bis-(2,5-difluorophenyl)-3-[(3-pyridyl)hydroxymethyl]thiophene, 2,4-Bis-(4-chloro-3-fluorophenyl)-3-[(3-pyridyl)hydroxymethyl]thiophene, 2,4-Bis-(3-methoxyphenyl)-3-[(3-pyridyl)hydroxymethyl]-thiophene, 4-(2-Fluorophenyl)-3-[(3-pyridyl)hydroxymethyl]-2-(2-thienyl)thiophene, 2,4-Bis-(2-chloro-4-trifluoromethylphenyl)-3-[(3-pyridyl)hydroxymethyl]thiophene, 2,4-Bis-(4-methoxyphenyl)-3-[(3-pyridyl)hydroxymethyl]thiophene, 2-(3-Chlorophenyl)-4-(2,4-difluorophenyl)-3-[(3-pyridyl)hydroxymethyl]-thiophene, 2-(5-Bromo-2-thienyl)-4-(2,4-difluorophenyl)-3-[(3-pyridyl)hydroxymethyl]thiophene, 2-(5-Chloro-2-thienyl)-4-(2,4-difluorophenyl)-3-[(3-pyridyl)hydroxymethyl]thiophene, 5-Chloro-2-(5-chloro-2-thienyl)-4-(2,4-difluorophenyl)-3-[(3-pyridyl)hydroxymethyl]-thiophene, 4-(4-Chlorophenyl)-2-(2-fluorophenyl)-3-[(3-pyridyl)hydroxymethyl]thiophene, 4-(4-Chlorophenyl)-2-(3-fluorophenyl)-3-[(3-pyridyl)hydroxymethyl]thiophene, 2-(2-Chlorophenyl)-4-(2,4-difluorophenyl)-3-[(3-pyridyl)hydroxymethyl]thiophene, 4-(2,4-Difluorophenyl)-2-(2-fluorophenyl)-3-[(3-pyridyl)hydroxymethyl]thiophene, 2-(4-Chlorophenyl)-4-(4-chloro-2-fluorophenyl)-3-[(3-pyridyl)hydroxymethyl]-thiophene, 2-(3-Chlorophenyl)-4-(4-chloro-2-fluorophenyl)-3-[(3-pyridyl)hydroxymethyl]-thiophene, and 4-(2,4-Difluorophenyl)-2-(4-fluorophenyl)-3-[(3-pyridyl)hydroxymethyl]thiophene;
and salts thereof.

43. The mixture of claim 31, wherein the compound of formula I is selected from the group consisting of:
2,4-Bis(2,4-difluorophenyl)-3-[(3-pyridyl)hydroxymethyl)thiophene and 2,4-Bis(4-chlorophenyl)-3-[(3-pyridyl)hydroxymethyl)thiophene;
and salts thereof.

44. The mixture of claim 31, wherein the compound II is an active compound selected from the group consisting of carbendazim, epoxiconazole, fluquinconazole, metconazole, propiconazole, prothioconazole, tebuconazole, triticonazole and prochloraz.

45. The mixture of claim 31, wherein the compound II is an active compound selected from the group consisting of azoxystrobin, trifloxystrobin, kresoxim-methyl, dimoxystrobin, orysastrobin, enestroburin, and metominostrobin.

46. The mixture of claim 31, wherein the compound II is an active compound selected from the group consisting of boscalid, metalaxyl, metalaxyl-M and dimethomorph.

47. The mixture of claim 31, wherein the compound II is an active compound selected from the group consisting of 5-ethyl-6-octyl-[1,2,4]triazolo[1,5-a]pyrimidine-7-ylamine, captan, cyprodinil, pyrimethanil, fenpropimorph, tridemorph, fenpropidin and iprodione.

48. The mixture of claim 31, wherein the compound II is an active compound selected from the group consisting of mancozeb, maneb, metam, metiram, thiram, propineb, iprovalicarb and thiophanate-methyl.

49. The mixture of claim 31, wherein the compound II is an active compound selected from the group consisting of dodine, dithianon, fosethyl-aluminium, phosphorous-acids and its salts, chlorothalonil, metrafenone and spiroxamine.

50. The mixture of claim 31, wherein the compound II is an active compound selected from the group consisting of dimoxystrobin, 5-ethyl-6-octyl-[1,2,4]triazolo[1,5-a]pyrimidine-7-ylamine, prothioconazole, iprovalicarb, chlorothalonil, carbendazim, propineb, fenpropimorph, tridemorph and tebuconazole.

51. The mixture of claim 31, further comprising an insecticidal compound.

52. The mixture of claim 51, wherein the insecticidal compound is selected from the group consisting of alpha-cypermethrin, acetamiprid, clothianidine, dinotefuran, imidacloprid, thiamethoxam, nitenpyram, and thiacloprid.

53. The mixture of claim 31, wherein compound I and compound II are present in a weight ratio of from 100:1 to 1:100.

54. A fungicidal composition, comprising a liquid or solid carrier and at least two fungicidally active ingredients of the mixture of claim 31.

55. A method for controlling phytopathogenic harmful fungi, wherein the fungi, the habitat, a plant to be protected against fungal attack, the soil, or a seed is treated with an effective amount of the mixture of claim 31.

56. The method of claim 55, wherein the mixture is applied in an amount of from 5 g/ha to 2000 g/ha.

57. The method of claim 55, wherein the compound of formula I and the compound II are applied simultaneously in a joint manner, simultaneously in a separate manner, or in succession.

58. The method for protection of seed, comprising contacting the seeds with a mixture of claim 31 in fungicidally effective amounts.

59. The method of claim 58, wherein the mixture is applied in an amount of from 0.01 g to 10 kg per 100 kg of seeds.

60. A seed comprising the mixture of claim 31 in an amount of from 0.1 g to 10 kg per 100 kg of the seed.
